(19) United States
(12) Patent Application Publication
(10) Pub. No.: US 2014/0205546 A1
(43) Pub. Date: Jul. 24, 2014

(54) NANOSCALE PARTICLE FORMULATIONS AND METHODS

(71) Applicant: Annuary Healthcare, Inc., La Jolla, CA (US)

(72) Inventor: John A. Macoviak, La Jolla, CA (US)

(21) Appl. No.: 14/143,824

(22) Filed: Dec. 30, 2013

Related U.S. Application Data
(63) Continuation of application No. 13/525,056, filed on Jun. 15, 2012, now abandoned.
(60) Provisional application No. 61/498,365, filed on Jun. 17, 2011.

(51) Int. Cl. A01N 59/16; A61K 9/00; A61K 33/38; A61K 9/70; A61K 9/14; A61K 33/24

(52) U.S. Cl. CPC A01N 59/16; A61K 9/14; A61K 33/24; A61K 33/38; A61K 9/7015; A61K 9/0014
USPC 424/10.3; 424/405; 424/617; 424/618

(57) ABSTRACT

Provided herein in some embodiments is a formulation comprising (a) one or more nanoscale particle; and (b) a film-forming polymer. In some instances, the formulation is an immediate and sustained released formulation suitable for topical administration or administration to surfaces. Also provided here in certain embodiments is a method of reducing the population of pathogenic microorganisms on skin or surfaces, the method comprising applying to the skin or surface a composition, the composition comprising (a) one or more nanoscale particle; and (b) a film-forming polymer.

### CROSS-REFERENCE

This application in a continuation of Ser. No. 13/525,056 filed Jun. 15, 2012, which claims the benefit of U.S. Provisional Application No. 61/498,365 filed Jun. 17, 2011, each of which is incorporated herein by reference in its entirety.

### BACKGROUND OF THE INVENTION

Microorganisms are responsible for a number of diseases and adverse conditions. It is generally understood that the majority of microbial pathogens (bacteria, fungi, yeast, molds, viruses, and protozoa) that cause disease gain entry into a mammal through various portals (eyes, ears, nose, mouth), and that these microbes are generally introduced into these portals by the hands. In addition, various types of microbial pathogens are acquired by direct contact with contaminated surfaces in the environment.

Materials and methods for preventing microbial contamination and infectious diseases are needed. A large number of illnesses may be prevented or alleviated by the decontamination of skin and surrounding surfaces.

The occurrence of healthcare-associated infections, also termed nosocomial infections, are of increasing concern both because of the danger posed to patients, as well as the significant direct costs imposed on hospitals and the healthcare system. The Centers for Disease Control and Prevention (CDC) estimates the annual cost of dealing with these cases to be between $1 to $1.6 billion, with other estimates proposing as much as $3 billion annually in costs related to bacterial infections (e.g., Clostridium difficile infection). Additionally, evidence suggests that both the number of occurrences and the severity of bacterial infection outbreaks are increasing.

Because of the rising prevalence and cost associated with the treatment of healthcare-associated infections, there is clearly a need for anti-microbial products that effectively kill the bacteria associated with these infections (e.g., C. difficile) and reduce the risk of outbreaks in healthcare facilities, ideally without the use of antibiotics or caustic chemicals.

### SUMMARY OF THE INVENTION

In accordance with the subject matter described herein, in certain embodiments the present disclosure provides a formulation comprising (a) one or more nanoscale particle; and (b) a film-forming polymer. Also provided here in certain embodiments is a method of reducing the population of pathogenic microorganisms on skin or surfaces, including for example hard surfaces. In some embodiments, the film-forming polymer enables the active agent to remain at surface of the skin or article for a longer period of time than it would otherwise remain without the film-forming polymer. As such, the formulations described herein provide for longer-lasting antimicrobial protection. In some instances, the formulation is an immediate and sustained released formulation suitable for topical administration or administration to surfaces.

Provided in certain embodiments herein is a topical formulation comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the formulation comprises at least one titanium dioxide nanoparticle, wherein the titanium dioxide nanoparticle has photocatalytic activity when irradiated with visible and ultraviolet light.

In some embodiments, provided herein is a formulation further comprising a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, provided herein is a formulation further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, provided herein is a formulation further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the formulation is in a form of an aerosol, cream, foam, emulsion, gel, liquid, lotion, mousse, patch, pomade, powder, solid, spray, stick, towelette, or any combinations thereof. In other embodiments, the formulation is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the formulation comprises at least one nanoscale particle, wherein the particle is present in a concentration of about 0.0000001% to 5%. In other embodiments, the formulation comprises a film-forming polymer, wherein the polymer is present in an amount of 0.05-5.0%.

In certain embodiments provided herein, the formulations described herein provide immediate release of at least one nanoscale particle. In further or alternative embodiments, the formulation provides sustained release of at least one nanoscale particle. In yet further or alternative embodiments, the formulation provides immediate and sustained release of at least one nanoscale particle.

Provided in some embodiments herein is an immediate and sustained release formulation suitable for topical administration comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the formulation comprises at least one titanium dioxide nanoparticle, wherein the titanium dioxide nanoparticle has photocatalytic activity when irradiated with visible and ultraviolet light. In some embodiments, the titanium dioxide oxidizes organic material (e.g. microbes).

In some embodiments, the formulation further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the formulation further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the formulation further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the formulation is in a form of an aerosol, cream, foam, emulsion, gel, liquid, lotion, mousse, patch, pomade, powder, solid, spray, stick, towelette, or any combinations thereof. In other embodiments, the formulation is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the formulation comprises at least one nanoscale particle, wherein the particle is present in a concentration of about 0.0000001% to 5%. In other embodiments, the formulation comprises a film-forming polymer, wherein the polymer is present in an amount of 0.05-5.0%.

Provide herein in some embodiments is a formulation suitable for application to a surface comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the formulation comprises at least one titanium dioxide nanoparticle, wherein the titanium dioxide nanoparticle has photocatalytic activity when irradiated with visible and ultraviolet light. In some embodiments, the titanium dioxide oxidizes organic material (e.g. microbes).

In some embodiments, the formulation further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the formulation further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the formulation further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the formulation is in a form of an aerosol, cream, foam, emulsion, gel, liquid, lotion, mousse, patch, pomade, powder, solid, spray, stick, towelette, or any combinations thereof. In other embodiments, the formulation is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the formulation comprises at least one nanoscale particle, wherein the particle is present in a concentration of about 0.0000001% to 5%. In other embodiments, the formulation comprises a film-forming polymer, wherein the polymer is present in an amount of 0.05-5.0%.

In certain embodiments provided herein, the formulations described herein provide immediate release of at least one nanoscale particle. In further or alternative embodiments, the formulation provides sustained release of at least one nanoscale particle. In yet further or alternative embodiments, the formulation provides immediate and sustained release of at least one nanoscale particle.

Provided herein in certain embodiments is a method of reducing the population of pathogenic microorganisms on skin for at least 6 hours comprising applying to the skin a composition, the composition comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the composition comprises at least one titanium dioxide nanoparticle, wherein the titanium dioxide nanoparticle has photocatalytic activity when irradiated with visible and ultraviolet light. In some embodiments, the titanium dioxide oxidizes organic material (e.g. microbes).

In some embodiments, the composition further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the composition further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the composition further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the composition is in a form of an aerosol, cream, foam, emulsion, gel, liquid, lotion, mousse, patch, pomade, powder, solid, spray, stick, towelette, or any combinations thereof. In other embodiments, the composition is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the composition comprises at least one nanoscale particle, wherein the particle is present in a concentration of about 0.0000001% to 5%. In other embodiments, the composition comprises a film-forming polymer, wherein the polymer is present in an amount of 0.05-5.0%.

In some embodiments, the pathogenic microorganisms are bacteria, viruses, fungi or combinations thereof. In specific embodiments, the pathogenic microorganisms are Aspergillus niger, Pseudomonas aeruginosa, Staphylococcus aureus, Clostridium difficile, and Vancomycin-Resistant Enterococci, H1N1 influenza virus, or combinations thereof.

In certain embodiments provided herein, the compositions described herein provide immediate release of at least one nanoscale particle. In further or alternative embodiments, the composition provides sustained release of at least one nanoscale particle. In yet further or alternative embodiments, the composition provides immediate and sustained release of at least one nanoscale particle.

Provided here in certain embodiments is a method of reducing the population of pathogenic microorganisms on a surface for up to 6 to 12 hours comprising applying to the surface a composition, the composition comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the composition comprises at least one titanium dioxide nanoparticle, wherein the titanium dioxide nanoparticle has photocatalytic activity when irradiated with visible and ultraviolet light.

In some embodiments, the composition further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the composition further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the composition further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the composition is in a form of an aerosol, cream, foam, emulsion, gel, liquid, lotion, mousse, patch, pomade, powder, solid, spray, stick, towelette, or any combinations thereof. In other embodiments, the composition is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the composition comprises at least one nanoscale particle, wherein the particle is present in a concentration of about 0.0000001% to 5%. In other embodiments, the composition comprises a film-forming polymer, wherein the polymer is present in an amount of 0.05-5.0%.

In some embodiments, the pathogenic microorganisms are bacteria, viruses, fungi or combinations thereof. In specific embodiments, the pathogenic microorganisms are Aspergillus niger, Pseudomonas aeruginosa, Staphylococcus aureus, Clostridium difficile, and Vancomycin-Resistant Enterococci, H1N1 influenza virus, or combinations thereof.

In certain embodiments provided herein, the compositions described herein provide immediate release of at least one nanoscale particle. In further or alternative embodiments, the composition provides sustained release of at least one nanoscale particle. In yet further or alternative embodiments, the composition provides immediate and sustained release of at least one nanoscale particle.

Provided herein in some embodiments is a method of killing at least one pathogenic microorganism on skin comprising applying to the skin a composition, the composition comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the composition comprises at least one titanium dioxide nanoparticle, wherein the titanium dioxide nanoparticle has photocatalytic activity when irradiated with visible and ultraviolet light. In some embodiments, the titanium dioxide oxidizes organic material (e.g. microbes).

In some embodiments, the composition further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the composition further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the composition further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the composition is in a form of an aerosol, cream, foam, emulsion, gel, liquid, lotion, mousse, patch, pomade, powder, solid, spray, stick, towelette, or any combinations thereof. In other embodiments, the composition is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the composition comprises at least one nanoscale particle, wherein the particle is present in a concentration of about 0.0000001% to 5%. In other embodiments, the composition comprises a film-forming polymer, wherein the polymer is present in an amount of 0.05-5.0%.

In some embodiments, the pathogenic microorganisms are bacteria, viruses, fungi or combinations thereof. In specific embodiments, the pathogenic microorganisms are Aspergillus niger, Pseudomonas aeruginosa, Staphylococcus aureus, Clostridium difficile, and Vancomycin-Resistant Enterococci, H1N1 influenza virus, or combinations thereof.

In certain embodiments provided herein, the compositions described herein provide immediate release of at least one nanoscale particle. In further or alternative embodiments, the composition provides sustained release of at least one nanoscale particle. In yet further or alternative embodiments, the composition provides immediate and sustained release of at least one nanoscale particle.

Provided herein in certain embodiments is a method of killing a pathogenic microorganism on a surface comprising applying to the surface a composition, the composition comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the composition comprises at least one titanium dioxide nanoparticle, wherein the titanium dioxide nanoparticle has photocatalytic activity when irradiated with visible and ultraviolet light.

In some embodiments, the composition further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the composition further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the composition further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the composition is in a form of an aerosol, cream, foam, emulsion, gel, liquid, lotion, mousse, patch, pomade, powder, solid, spray, stick, towelette, or any combinations thereof. In other embodiments, the composition is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the composition comprises at least one nanoscale particle, wherein the particle is present in a concentration of about 0.0000001% to 5%. In other embodiments, the composition comprises a film-forming polymer, wherein the polymer is present in an amount of 0.05-5.0%.

In some embodiments, the pathogenic microorganisms are bacteria, viruses, fungi or combinations thereof. In specific embodiments, the pathogenic microorganisms are Aspergillus niger, Pseudomonas aeruginosa, Staphylococcus aureus, Clostridium difficile, and Vancomycin-Resistant Enterococci, H1N1 influenza virus, or combinations thereof.

In certain embodiments provided herein, the compositions provided herein facilitate immediate release of at least one nanoscale particle. In further or alternative embodiments, the composition provides sustained release of at least one nanoscale particle. In yet further or alternative embodiments, the composition provides immediate and sustained release of at least one nanoscale particle.

In certain embodiments, the surface is substantially free of microorganisms for at least 24 hours. In some embodiments, the surface is substantially free of microorganisms for about 6 hours to 1 week, 6 hours to 3 days, 12 hours to 2 days, or about 24 hours. In further or additional embodiments, the surface is smooth or non-porous. In specific embodiments, the smooth or non-porous surface is substantially free of microorganisms for at least 24 hours. In still specific embodiments, the smooth or non-porous surface is substantially free of microorganisms for about 6 hours to 1 week, 6 hours to 3 days, 12 hours to 2 days, or about 24 hours. In other embodiments, the surface is substantially free of microorganisms for about 1 year. In further or additional embodiments, the surface is substantially free of microorganisms for about 1 week to 5 years, 1 month to 2 years, 2 months to 1.5 years, 3 months to 1 year, or for about 6 months. In yet further or alternative embodiments, the surface is porous. In specific embodiments, the porous surface is substantially free of microorganisms for about 1 year. In still specific embodiments, the porous surface is substantially free of microorganisms for about 1 week to 5 years, 1 month to 2 years, 2 months to 1.5 years, 3 months to 1 year, or for about 6 months. In some embodiments, the surface is a medical device. In certain specific embodiments, the medical device is an IV catheter, heart valve, or pacemaker.

Provided herein in certain embodiments is a biocide comprising: (a) one or more nanoscale particle having a particle size between 1 and 100 nm or 5 and 200 nm; and (b) a film-forming polymer.

In some embodiments, the film-forming polymer is selected from polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In specific embodiments, the film-forming polymer is polyolprepolymer-2 (PPG-12/SMDI).

In some embodiments, at least one nanoscale particle is silver, titanium, zinc, aluminum, iron, copper, platinum, zirconium, palladium, gold, salts thereof or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the composition comprises at least one titanium dioxide nanoparticle, wherein the titanium dioxide nanoparticle has photocatalytic activity when irradiated with visible and ultraviolet light.

In some embodiments, the biocide further comprises a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, the biocide further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, the biocide further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the biocide is in a form of an aerosol, cream, foam, emulsion, gel, liquid, lotion, mousse, patch, pomade, powder, solid, spray, stick, towelette, or any combinations thereof. In other embodiments, the biocide is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the biocide comprises at least one nanoscale particle, wherein the particle is present in a concentration of about 0.0000001% to 5%. In other embodiments, the composition comprises a film-forming polymer, wherein the polymer is present in an amount of 0.05-5.0%.

In some embodiments, the pathogenic microorganisms are bacteria, viruses, fungi or combinations thereof. In specific embodiments, the pathogenic microorganisms are Aspergillus niger, Pseudomonas aeruginosa, Staphylococcus aureus, Clostridium difficile, and Vancomycin-Resistant Enterococci, H1N1 influenza virus, or combinations thereof.

In certain embodiments provided herein, the biocides described herein provide immediate release of at least one nanoscale particle. In further or alternative embodiments, the biocide provides sustained release of at least one nanoscale particle. In yet further or alternative embodiments, the biocide provides immediate and sustained release of at least one nanoscale particle.

Provided in some embodiments is a topical formulation comprising: (a) one or more nanoscale particle having a particle size between 1 and 100 nm or 5 and 200 nm; and (b) polyolprepolymer-2 (PPG-12/SMDI).

In some embodiments, at least one nanoscale particle is silver, titanium, zinc, aluminum, iron, copper, platinum, zirconium, palladium, gold, or combinations thereof. In other embodiments, at least one nanoscale particle comprises silver, titanium, zinc, aluminum, iron, copper, platinum, zirconium, palladium, or gold. In certain embodiments, at least one nanoparticle is zinc oxide, copper oxide, iron oxide, aluminum oxide, or platinum oxide. In certain embodiments, the silver nanoparticle is an ionic silver salt or elemental silver. In further or additional embodiments, the elemental silver is colloidal silver. In yet further or additional embodiments, the ionic silver salt is silver chloride, silver bromide, silver phosphate, silver nitrate, silver citrate, or combinations thereof. In further or alternative embodiments, the nanoscale particle is titanium dioxide. In still further or alternative embodiments, the titanium dioxide is in an anatase phase. In certain specific embodiments, the formulation comprises at least one titanium dioxide nanoparticle, wherein the titanium dioxide nanoparticle has photocatalytic activity when irradiated with visible and ultraviolet light.

In some embodiments, provided herein is a formulation further comprising a surfactant. In certain embodiments, the surfactant is benzalkonium chloride, benzethionium chloride, cetyl trimethyl ammonium chloride, trimethyl coco quaternary ammonium chloride, diquaternary polydimethylsiloxane, or trimethylquaternary ammonium chloride.

In certain embodiments, provided herein is a formulation further comprises an iodine source. In specific embodiments, the iodine source is iodine, tincture of iodine, iodine salts, povidone-iodine, or combinations thereof. In certain specific embodiments, the iodine source is povidone-iodine.

In some embodiments, provided herein is a formulation further comprises a vehicle acceptable for topical administration. In specific embodiments, the vehicle is tap water, de-ionized water, distilled water, aqueous solvent system, aqueous-based single phase liquid solvent system, hydro-alcoholic solvent system, glycerin, anhydrous liquid solvent, oil, alcohol, or any combinations thereof. In further or additional embodiments, the vehicle is ethyl alcohol.

In some embodiments, the formulation is in a form of an aerosol, cream, foam, emulsion, gel, liquid, lotion, mousse, patch, pomade, powder, solid, spray, stick, towelette, or any combinations thereof. In other embodiments, the formulation is an oral care product, over-the-counter drug, over-the-counter pharmaceutical, suncare product, sunscreen product, foot-care product, liquid and bar soap, cleaning product, antiperspirant product, deodorant product, fragrance product, insect repellant, cosmetic product, hair care product, shampoo, hair conditioner, hair spray or moisturizer.

In some embodiments, the formulation comprises at least one nanoscale particle, wherein the particle is present in a concentration of about 0.0000001% to 5%. In other embodiments, the formulation comprises a film-forming polymer, wherein the polymer is present in an amount of 0.05-5.0%.

In certain embodiments provided herein, the formulations described herein provide immediate release of at least one nanoscale particle. In further or alternative embodiments, the formulation provides sustained release of at least one nanoscale particle. In yet further or alternative embodiments, the formulation provides immediate and sustained release of at least one nanoscale particle.

In some embodiments, the formulation further comprises a moisturizer. In specific embodiments, the formulation further comprises a moisturizing agent in a concentration of about 0.5% to about 15%, about 5% to about 15%, about 10% to about 25%, about 10% to about 50%, about 10% to about 75%, about 10% to about 95%, about 0.5% to about 95%, about 5% to about 75%, about 15% to about 75%, about 25% to about 75%, about 50% to about 75%, about 15% to about 25%, about 15% to about 50%, greater than about 1%, greater than about 5%, greater than about 10%, greater than about 20%, or greater than about 50%.

In further or additional embodiments, the formulation further comprises a coloring agent that adheres to the skin during use to indicate compliant application of product. In specific embodiments, the color of the coloring agent gradually fades to indicate the necessity for reapplication of the product.

### DETAILED DESCRIPTION OF THE INVENTION

While preferred embodiments have been shown and described herein, it will be apparent to those skilled in the art that such embodiments are provided by way of example only. Numerous variations, changes, and substitutions will now occur to those skilled in the art without departing from the spirit of the disclosure. It should be understood that various alternatives to the embodiments described herein may be employed in practicing the subject matter described herein. It is intended that the following claims define the scope of the disclosure and that methods and structures within the scope of these claims and their equivalents be covered thereby.

Provided herein in some embodiments is a formulation comprising (a) one or more nanoscale particle; and (b) a film-forming polymer. In some instances, the formulation is an immediate and sustained released formulation suitable for topical administration or administration to surfaces. Also provided here in certain embodiments is a method of reducing the population of pathogenic microorganisms on skin or surfaces, the method comprising applying to the skin or surface a composition, the composition comprising (a) one or more nanoscale particle; and (b) a film-forming polymer. Certain embodiments provide a method of killing at least one pathogenic microorganism on the skin or surface, the method comprising applying to the skin or surface a composition, the composition comprising (a) one or more nanoscale particle; and (b) a film-forming polymer. In some embodiments, the formulations and compositions described here are odor control agents, antimicrobial surface coatings, self-cleaning surface coatings, germicide, antibacterial agents, anti-microbials, antifungals, anti-viral agents, anti-protozoal agents, microbiostats, or disinfectants.

### CERTAIN DEFINITIONS

The term “nanoscale particle” includes nanospheres, nanorods, nanofibers, and nanowires. In some embodiments, these nanoscale particles are part of a nano-network.

The term “average” dimension of a plurality of nanoparticles means the average of that dimension for the plurality.

The term “photocatalysis” means catalysis that is dependent on the presence of electromagnetic radiation to catalyze a reaction.

The term “visible light” means electromagnetic radiation having a wavelength from 380 nm to 780 nm.

In some embodiments, the combination compositions disclosed herein act additively or synergistically. In some embodiments, a “synergistic effect” is seen where the combination of the nanoscale particle and additional active agent(s) results in an activity that is more than the effect of the two individual agents alone.

As used in the specification and the appended claims, the singular forms “a”, “an” and “the” include plural references unless the context clearly dictates otherwise. Thus for example, reference to “the method” includes one or more methods, and/or steps of the type described herein and/or which will become apparent to those persons skilled in the art upon reading this disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “alkyl” as used herein refers to saturated or unsaturated, straight- or branched-chain hydrocarbon radicals derived from a hydrocarbon moiety containing between one and twenty carbon atoms by removal of a single hydrogen atom. Alkyl groups as used herein optionally include one or more further substituent groups. This term is exemplified by groups such as methyl, ethyl, n-propyl, isopropyl, n-butyl, iso-butyl, tert-butyl, n-hexyl, n-octyl, tert-octyl and the like, and are substituted or unsubstituted. The term “lower alkyl” refers to alkyl groups having 1 to 6 carbon atoms. The term “alkyl” also includes “cycloalkyls” as defined below.

“Subject” includes humans. The terms “human,” “patient” and “subject” are used interchangeably herein.

“Effective amount” means the amount of a compound that, when administered to a subject for treating a disease, cosmetic or dermatological condition, is sufficient to effect such treatment for the disease, cosmetic or dermatological condition. The “effective amount” can vary depending on the compound, the disease and its severity, and the age, weight, etc., of the subject to be treated.

### Nanoscale Particles

Some embodiments of the present disclosure describe a formulation or composition comprising at least one nanoscale particle. In some embodiments, the nanoscale particles comprise silver, titanium, zinc, aluminum, iron, copper, platinum, zirconium, palladium, gold, manganese, mercury, magnesium, silica, chromium, cobalt, nickel, molybdenum, ruthenium, rhodium, cadmium, cesium, iridium, osmium, tungsten, selenium, antimony, tin, cerium, yttrium, samarium, lanthanum, gallium, erbium, bismuth, strontium, barium, arsenic, salt thereof, or combinations thereof. In certain specific embodiments, the nanoparticle comprises zinc oxide, copper oxide, iron oxide, aluminum oxide, platinum oxide, zirconium oxide, yttrium oxide, colloidal gold, an ionic silver salt, elemental silver, titanium dioxide, bismuth pyrithione, zinc pyrithione, zinc percarbonates, zinc perborates, bismuth salts, arsenic trioxide, arsenicals, or combinations thereof.

In some embodiments, the nanoparticle comprises colloidal silver, metallic silver, silver chloride, silver bromide, silver phosphate, silver nitrate, silver citrate, silver acetate, silver benzoate, silver pyrithione, or combinations thereof. Silver nanoparticles having a particle size of about 1 to about 100 nm or 5 and 200 nm are generally understood to slowly release antimicrobial silver ions (e.g., Ag⁺).

In some embodiments, the release rate of metal ions (e.g., silver ions) depends on the initial concentration and size of the nanoscale particles. In certain embodiments, the release rate of metal ions (e.g., silver ions) is an indicator of the biocidal activity of the nanoscale particles. For example, in some instances, silver nanoscale particles in an aqueous environment oxidize in the presence of oxygen and protons according to the stoichiometric reaction:

Ag₍ₛ₎+½O₂+2H_{(aq)}⁺ [symbol] 2Ag_{(aq)}⁺+H₂O_{(I)}

releasing Ag+ ions during particle dissolution.

In further or alternative embodiments, the nanoparticle comprises titanium dioxide in the anatase phase. In some embodiments, the nanoscale titanium dioxide is photocatalytically active. Titanium dioxide in the anatase phase is generally understood to promote oxidation-reduction (redox) reactions when irradiated with ultraviolet or visible light. In some embodiments, a nanoparticle containing titanium dioxide that is irradiated with visible or ultraviolet light in an aqueous environment (e.g., within a microorganism) and in certain situations produce hydroxyl ions (OH⁻), superoxide ions (O₂⁻), and/or hydrogen peroxide (H₂O₂). In additional or further embodiments, a nanoscale particle containing titanium dioxide that is exposed to visible light while in a cell or in contact with a cell produces a toxic environment and damages or kills the cell. In some embodiments, the titanium dioxide oxidizes organic material (e.g. microbes).

In certain embodiments, suitable copper nanoscale particles include cupric oxide, cuprous oxide, cuprous iodide, cupric iodide, cupric phosphate, copper (II) hydrogen phosphate, and cupric silicate.

In some embodiments, the nanoscale particles have an average particle size between 0.1 and 500 nm, 0.1 and 400 nm, 0.1 and 300 nm, 0.1 and 250 nm, 0.1 and 200 nm, 0.1 and 100 nm, 0.1 and 90 nm, 1 and 500 nm, 1 and 450 nm, 1 and 400 nm, 1 and 350 nm, 1 and 300 nm, 1 and 250 nm, 1 and 225 nm, 1 and 200 nm, 1 and 175 nm, 1 and 150 nm, 1 and 125 nm, 1 and 100 nm, 1 and 75 nm, 1 and 50 nm, 1 and 40 nm, 1 and 30 nm, 1 and 25 nm, 1 and 20 nm, 1 and 15 nm, or 1 and 10 nm. In some embodiments, the nanoscale particles have an average particle size between 5 and 10 nm, 5 and 30 nm 10 and 250 nm, 10 and 200 nm, 50 and 200 nm, or 100 and 200 nm. In further or additional embodiments, the nanoscale particles have an average particle size of less than about 500 nm, less than about 450 nm, less than about 400 nm, less than about 350 nm, less than about 300 nm, less than about 250 nm, less than about 200 nm, less than about 175 nm, less than about 150 nm, less than about 125 nm, less than about 100 nm, less than about 95 nm, less than about 90 nm, less than about 85 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 28 nm, less than about 25 nm, less than about 23 nm, less than about 20 nm, less than about 18 nm, less than about 15 nm, or less than about 10 nm. In further or additional embodiments, the nanoscale particles have an average particle size of about 500 nm, about 400 nm, about 350 nm, about 300 nm, about 250 nm, about 200 nm, about 175 nm, about 150 nm, about 125 nm, about 100 nm, about 75 nm, about 60 nm, about 50 nm, about 25 nm, or about 10 nm. In some embodiments, the formulation comprises a bimodal distribution of particle sizes. In some embodiments, the formulation comprises a polydisperse population of particle sizes.

In some instances, for cases with similar silver concentrations by mass, samples with smaller particle size have a larger number concentration. In some instances, the increase in concentration of silver nanocrystals leads to a higher nanoparticle:bacterium ratio and a greater amount of available nanoparticle surface area.

In some embodiments, the rate of release of Ag+ ions is dependent upon the size of the nanocrystal, with smaller particles dissolving more readily. In some instances, the rate of release of nanoscale ions (e.g., silver nanoscale ions) is inversely proportional to the size of the nanoscale particle (e.g., nanoscale colloidal silver).

Some embodiments provided herein describe nanoscale particles having a diversity of shapes. In some embodiments, the nanoscale particles are spherical in shape. In other embodiments, the nanoscale particles are round plates, triangular plates, square plates, or hexagonal plates. In certain embodiments, the nanoscale particles are triangular plates. In some embodiments, the nanoscale particles are nanorods, hexagonal-shaped, cube-shaped, polyhedron-shaped, or star-shaped. In some embodiments, the formulation comprises a bimodal distribution of particle shapes. In some embodiments, the formulation comprises a polydisperse population of particle shapes.

In some embodiments, the nanoscale particles comprise composites. Non limiting examples of suitable nanoscale particles include alloy of silver containing about 2.5 wt % copper, alumina-silver nanoscale composite, titania-silver nanoscale composite, silver-copper nanoscale composite, silver-iron oxide nanoscale composite, silver-silica nanoscale composite, and silver-selenium nanoscale composite. In some embodiments, the formulation comprises a nanoparticle/polymer composite film (e.g., silver nanoparticle/polyvinylpyrrolidone (PVP), silver nanoparticle/polyvinyl alcohol (PVA), etc.)

Some embodiments provided herein describe one ore more nanoscale particles optionally comprising a capping ligand or stabilizing agent. Non-limiting examples of capping or stabilizing agents include polyvinylpyrrolidone (PVP), polyethyleneimine, citrate, keratin, tannic acid, bovine serum albumin (BSA), ionic surfactants (e.g, sodium dodecyl sulfate (SDS)), non-ionic surfactants (e.g., Tween 80), linoleic acid, poly(methylvinylether-co-maleic anhydride (PVM/MA), and sophorolipid.

In some embodiments, the nanoscale particle has antimicrobial activity. In further or additional embodiments, the nanoscale particle is an active agent. In still further or additional embodiments, the nanoscale particle is an odor control agent, antimicrobial surface coating, self-cleaning surface coating, germicide, antibacterial agent, anti-microbial, anti-fungal, anti-viral agent, anti-protozoal agent, microbiostat, or disinfectant.

### Film-Forming Polymer

In some embodiments, the formulation or composition comprises a film-forming polymer. In some instances, the film-forming polymer leaves a protective film on the surface of the skin either immediately or upon evaporation of volatiles in the composition. In further embodiments, the film-forming polymer improves the water-, sweat-, transfer- and wear-resistance properties of the formulation or composition. In certain aspects, the film-forming polymer enhances the spread characteristics of the composition, which allows the composition to be more uniformly and consistently applied to skin or an article surface. In some embodiments, the film-forming polymer improves smoothness of the formulation. In some instances, the film-forming polymer is a penetration enhancer.

In further or additional embodiments, when used with an one or more active agent(s), the film-forming polymer maintains the active agent at the surface of the skin or article for a longer period of time than it would otherwise remain without the film-forming polymer. In some embodiments, the film-forming polymer affords controlled release of the one or more active agent(s). In further or additional embodiments, the film-forming polymer affords sustained release of the one or more active agent(s). In further or alternative embodiments, the film-forming polymer affords immediate release of the one or more active agent(s). In further or alternative embodiments, the film-forming polymer affords immediate and controlled release of the one or more active agent(s). In yet further or additional embodiments, the film-forming polymer affords sustained release of the one or more active agent(s).

In certain embodiments, the film-forming polymer (e.g., polyprepolymer) suspends the antimicrobial (e.g., nanoparticle) to form a long lasting liquid reservoir in the stratum corneum and epidermis. In further or additional embodiments, the film-forming polymer (e.g., polyprepolymer) significantly influences the deposition of the antimicrobial (e.g., nanoparticle) on the surface of the skin or hard surface. In certain embodiments, the antimicrobial (e.g., nanoparticle) and film-forming polymer (e.g., polyprepolymer) remain on the surface of the skin or hard surface and does not penetrate. In some embodiments, the antimicrobial (e.g., nanoparticle) is suspended and remains active on the surface of the skin where it is most efficacious. In some embodiments, the film-forming polymer (e.g., polyprepolymer) provides an increased bioavailability and/or safety profile with respect to the antimicrobial formulation. In some embodiments, the film-forming polymer (e.g., polyprepolymer) prevents agglomeration of the antimicrobial (e.g., nanoparticle). In some embodiments, the film-forming polymer prevents protein binding (e.g., non-specific protein binding) of the microbial (e.g., nanoparticle). The formulations and compositions described herein provide a more safe and efficacious advancement in the art compared to existing technologies.

In some embodiments, the film-forming polymer is a synthetic polymer, a polymer of natural origin or mixture thereof.

Examples of film-forming polymers include, but are not limited to, one or more acrylate copolymers such as acrylate/octylacrylamide copolymers and acrylate/vinyl acetate copolymers; cellulosic polymers such as methyl cellulose and hydroxyethyl cellulose; ethylene/acrylic acid copolymer; polyacrylic acid; C₁ to C₅ alkyl galactomannan; isododecane/ethylene mixed copolymer; adipic acid/diethylene glycol/glycerin crosspolymer; trimethylpentanediol/adipic acid copolymer; trimethylpentanediol/adipic acid/isononanoic acid; polyvinyl pyrrolidone copolymer, PVP/hexadecene copolymer (e.g., Ganex V-216); PVP/eicosene copolymer (e.g., Ganex V-220); PVP/tricontanyl copolymer (e.g., Ganex WP-660); PVP/vinyl acetate copolymer; allyl stearate/vinyl acetate copolymers; alpha olefin/isopropyl maleate/MA polymer; cycloalkyl methacrylate copolymer/isododecane trimethyl polysiloxane; octadecene/MA copolymer; polypropylene glycol/sodium maleic acid diisobutylene copolymers (e.g., PPG-12/SMDI copolymer and PPG-51/SMDI, available from Penederm Inc.); poly(styrene-co-maleic anhydride) copolymers (SMA); acrylates C₁₀ to C₃₀ alkyl acrylate crosspolymer; cetyl hydroxyethylcellulose; dimethiconol; dimethicone; diglycol/cyclohexane-dimethanol/isophthalates/sulfoisophthalate copolymer; polyethylene; ethoxydiglycol; waxes such as beeswax and botanical waxes; polyurethane, polyurethane resins; natural gums; or any combinations of these ingredients. The polyurethane resins include Polyurethane-1, Polyurethane-2, Polyurethane-4, Polyurethane-5, and mixtures thereof. Additional film formers include those set forth in U.S. Pat. No. 5,916,541, which is incorporated herein by reference.

In some embodiments, the film-forming polymer comprising polyolprepolymer-2 (PPG-12/SMDI), polyolprepolymer-14 (PPG-51/SMDI), poly(styrene-co-maleic anhydride) copolymers (SMA); acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or any combination thereof. In some embodiments, any suitable film-forming polymer is used.

In certain embodiments, the film-forming polymer is an oil soluble penetration enhancer. In other embodiments, the film-forming polymer is a water soluble penetration enhancer.

### Additional Active Agents

In some embodiments, the present composition optionally includes one or more of the following additional ingredients: anesthetics, anti-allergenics, antifungals, antimicrobials, anti-inflammatories, antiseptics, chelating agents, colorants, depigmenting agents, emollients, exfollients, fragrances, humectants, lubricants, moisturizers, pharmaceutical agents, preservatives, skin protectants, skin penetration enhancers, stabilizers, surfactants, thickeners, viscosity modifiers, and vitamins.

In some embodiments, any formulation or composition described herein further comprises a source of iodine. Non-limiting examples of a source of iodine include iodine, iodophors, tincture of iodine, iodine salts, povidone-iodine, and combinations thereof. In some embodiments, any formulation or composition described herein further comprises any suitable iodophor.

In some embodiments, any formulation or composition described herein further comprises a surfactant. Examples of surfactants include but are not limited to quaternary ammonium salt benzalkonium ions (e.g., benzalkonium chloride), poly(ethyleneimine), DAXAD 19 (distributed by GEO Specialty Chemicals), benzethionium chloride, cetyl trimethyl ammonium chloride, trimethyl coco quaternary ammonium chloride, diquaternary polydimethylsiloxane, tris(2-hydroxyethylamine) benzyl ammonium chloride, monoalkyltrimethylammonium salts, dialkyldimethylammonium salts, heteroaromatic ammonium salts, polysubstituted quaternary ammonium salts, bis-quaternary ammonium salts, and polymeric quaternary ammonium salts, cocamidopropyldimethyl betaine, and trimethylquaternary ammonium chloride. In some embodiments, the surfactant is n-alkyl dimethylbenzylalkonium chloride, wherein said n-alkyl group is 10 to 20, 10 to 18, 10 to 16, 12 to 16, or 10 to 14 carbons in length. In some embodiments, the surfactant is Stepanquat® 50 NF. In certain embodiments, the surfactant has antimicrobial properties. In some embodiments, the formulation or composition further comprises any quaternary amines suitable for a bactericide. In some embodiments, the surfactant is a benzalkonium homolog having the structure of:

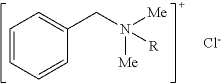

wherein R is an alkyl chain of from 10 to 17, 10 to 15, 10 to 18, 10 to 16, 12 to 16, or 10 to 14 carbon atoms. Non-limiting examples of these homologs include N,N-dimethyldecylammonium chloride, N,N-dimethylundecylammonium chloride, N,N-dimethyldodecylammonium chloride, N,N-dimethyltridecylammonium chloride, N,N-dimethyltetradecylammonium chloride, N,N-dimethylpentadecylammonium chloride, N,N-dimethylhexadecylammonium chloride, N,N-heptadecylammonium chloride, and combinations thereof.

In certain embodiments, any composition or formulation described herein further comprises a coloring agent, photochromic agent or photoactive agent. In certain embodiments, a coloring agent or photochromic pigment provides as a visual signal or indicator of antimicrobial protection. In some embodiments, a color change or color fade indicates to the user that re-application is necessary to maintain antimicrobial protection. In further or additional embodiments, the coloring agent provides a color change or fade after 0.5 h, 1 h, 2 h, 3 h, 6 h, 12 h, 15 h, 18 h, 24 h, 36 h, 48 h, 3 days, 4 days, 5 days, 6 days, 1 week, 2 weeks, 3 weeks, 1 month, 2 months, 3 months, 4 months, 5, months, 6 months, 7 months, 8 months, 9 months, 10 months, 11 months, 1 year, or 2 years. In some embodiments, the present composition is clear or colorless when applied, and becomes colored when re-application is necessary to maintain antimicrobial protection. In further or alternative embodiments, the present composition is colored when applied, then becomes colorless, clear or faded when re-application is necessary to maintain antimicrobial protection. In certain embodiments, the present composition is clear or colorless in outdoor light, and becomes colored in indoor light. For example, when used in medical and/or pharmaceutical applications, the compositions described herein are topically applied as an anti-microbial during outpatient surgery to indicate duration/wear of the anti-microbial. The applied composition remains colored during the length of the surgery to indicate that, for example, the anti-microbial is still taking effect. As the color intensity begins to fade, the surgeon knows that the effect of the anti-microbial is beginning to wear off. However, even if the surgery is completed before the anti-microbial composition or formulation wears off, the composition becomes clear or colorless when the person exits the outpatient facility and is exposed to outdoor light. In some instances, the coloring agent or photochromic material is colorless at a light intensity greater that about 1 Joule/cm², preferably greater than about 2 Joules/cm², and most preferably greater than about 5 Joules/cm². In other instances, the coloring agent or photochromic material is colored at a light intensity less than about 5 Joules/cm², preferably less that about 2 Joules/cm² and most preferably less that about 1 Joule/cm².

In some embodiments, organic photochromic compounds that are used in the present compositions and formulations include, but are not limited to azobenzene compounds, thioindigo compounds, dithizone metal complexes, spiropyran compounds, spirooxazine compounds, napthopyran compounds, fulgide compounds, dihydropyrene compounds, spirothiopyran compounds, 1,4-2H-oxazine, triphenylmethane compounds, viologen compounds, or any combinations thereof. In specific embodiments, organic photochromic compounds that are used in the present compositions and formulations include, but are not limited to, 1,3,3-trimethylspiro[indolino-2,3′(3H)naphtho(2,1-b)(1,4,)-oxazine]; 5-methoxy-1,3,3-trimethylspiro[indolino-2,3′-(3H)naptho(2,1-b)(1,4)-oxazine]; 5-chloro-1,3,3-trimethylspiro[indolino-2,3′-(3H)naphtho(2,1-b)(1,4)-oxazine]; 8′-piperidino-1,3,3-trimethylspiro[indolino-2,3′-(3H)naphtho(2,1-b)(1,4)-oxazine]; 1-benzyl-3,3-dimethyspiro[indolino-2,3′-d(3H)naphtho(2,1-b)(1,4)-oxazine]; 1,3,5,6,-tetramethyl-3-ethylspiro[indolino-2,3′-(3H)naphtho(2,1-b)(1,4)-oxazine]; 1,3,3,5,6-pentamethylspiro[indolino-2,3′-(3H)naphtho(2,1-b)(1,4)oxazine]; 1,3′,3′-trimethylspiro(2H-1benzopyran-2,2′-indolino); 3,3,1-diphenyl-3H-naphtho-(2,1,1-b)pyran; 1,3,3-triphenylspiro[indolino-2,3′-(3H)naphtho(2,1-b)pyran]; 1-(2,3,4,5,6-pentamethylbenzyl)-3,3-dimethylspiro[indolino-2,3′-(3H)-naphtho(2,1-b)pyran]; 1-(2-nitrobenzyl)-3,3-dimethylspiro[indolino-2,3′-(3H)-naphtho(2,1-b)pyran]; 1,1-diphenylnaphthopyran, 2,5-dimethylfuryl-trimethyfulgide, 2-methyl-5-chlorotrimethylfulgide, or any combinations thereof. In certain embodiments, photochromic and thermochromic material are used in combination.

In certain embodiments, any composition or formulation described herein further comprises one or more moisturizing agent. In specific embodiments, moisturizing agents that are used in the present compositions and formulations include, but are not limited to various polyethylene glycols (e.g., PEG 4, PEG 6, PEG 8, PEG 12 and PEG 20), sorbitol, propylene glycol monostearate, glycerin, fatty acid esters of α-tocopherol (e.g., linoleic acid ester of α-tocopherol, oleic acid ester of α-tocopherol, linolenic acid ester of α-tocopherol, palmitic acid of α-tocopherol, stearic acid ester of α-tocopherol, and myristic acid ester of α-tocopherol), oils (e.g., mineral oil, carob bean oil, palm oil, cabbage palm oil, coconut oil, jojoba oil, sunflower seed oil, high oleic sunflower oil, grapeseed oil, black mustard oil, ocilet oil, shea butter, sweet almond oil, soya-bean oil, avocado oil, peanut oil, cottonseed oil, sesame oil, olive oil, maize oil, coconut butter, castor oil, Ben oil, linseed oil, colza oil, annato oil, cornseed oil, safflower oil, walnut oil, hazelnut oil rapeseed oil, horsehair oil, mink oil, turtle oil, whale oil, fish oil, fish-liver oil, soft-roe oil, neat's-foot oil, tallows and egg oil), aloe extracts, mucopolysaccharides, collagen, lecithin, squalene, panthenol (e.g., D-panthenol), Hydromide® Blend, Liponate® GC, vitamin D3, ceramide, pseudoceramide, phytosterols (e.g., Net Sterol 100), hyaluronic acid, sodium hyaluronate, xylitolglucoside, xylitol, anhydroxylitol, sodium pyrollidone carboxylate, sodium lactate, orotic acid, propylene glycol, honey, petrolatum, lanolin, silicones (e.g., dimethicone), fatty acids, fatty acid esters, cholesterol, keratin and elastin.

In yet other embodiments, the composition comprises at least one sunscreen, sunprotectant or sunblock agent. “Sunscreen”, “sunprotectant” or “sunblock” as used herein defines ultraviolet ray-blocking compounds exhibiting absorption or blockage within the wavelength region between about 290 and 420 nm. Such agents are classified into five groups based upon their chemical structure: para-amino benzoates; salicylates; cinnamates; benzophenones; and miscellaneous chemicals including menthyl anthralinate and digalloyl trioleate. Inorganic sunscreens that are optionally used include titanium dioxide, zinc oxide, iron oxide and polymer particles such as those of polyethylene and polyamides. Specific suitable sunscreen agents include, for example: p-aminobenzoic acid, its salts and its derivatives (ethyl, isobutyl, glyceryl esters; p-dimethylaminobenzoic acid); Anthranilates (i.e., o-aminobenzoates; methyl, menthyl, phenyl, benzyl, phenylethyl, linalyl, terpinyl, and cyclohexenyl esters); Salicylates (amyl, phenyl, benzyl, menthyl, glyceryl, and dipropylene glycol esters); Cinnamic acid derivatives (methyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,3-disulfonic and of 2-naphthol-6,8-disulfonic acids); Dihydroxynaphthoic acid and its salts; o- and p-Hydroxybiphenyidisulfonates; Coumarin derivatives (7-hydroxy, 7-methyl, 3-phenlyll); Diazoles (2-acetyl-3-bromoindazole, phenyl benzoxazole, methyl naphthoxalole, various aryl benzothiazoles); Quinine salts (bisulfate, sulfate, chloride, oleate, and tannate); quinoline derivatives (8-hydroxyquinoline salts, 2-phenylquinoline); Hydroxy or methoxy substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzene, Sulisobenzone, Dioxybenzone, Benzoresorcinol, 2,2′,4,4′-Tetrahydroxybenzophenone, 2,2′-Dihydroxy4,4′-dimethoxybenzophenone, Octabenzone; 4-Isopropyhldibenzoylmethane; Butylmethoxydibenzoylmethane; Etocrylene; and 4-isopropyl-di-benzoylmethane; titanium dioxide, iron oxide, zinc oxide, and mixtures thereof. Other cosmetically-acceptable sunscreens and concentrations (percent by weight of the total cosmetic sunscreen composition) include diethanolamine methoxycinnamate (10% or less), ethyl-[bis(hydroxypropyl)]aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less). Yet other cosmetically-acceptable sunscreens and concentrations (reported as a percentage by weight of the total cosmetic sunscreen composition, and referring to the final percentage of the sunscreen) include: aminobenzoic acid (also called para-aminobenzoic acid and PABA; 15% or less; a UVB absorbing organic sunscreen), avobenzone (also called butyl methoxy dibenzoylmethane; 3% or less, a UVA I absorbing organic sunscreen), cinoxate (also called 2-ethoxyethyl p-methoxycinnamate; 3% or less, a UVB absorbing organic sunscreen), dioxybenzone (also called benzophenone-8; 3% or less, a UVB and UVA II absorbing organic sunscreen), homosalate (15% or less, a UVB absorbing organic sunscreen), menthyl anthranilate (also called menthyl 2-aminobenzoate; 5% or less, a UVA II absorbing organic sunscreen), octocrylene (also called 2-ethylhexyl-2-cyano-3,3 diphenylacrylate; 10% or less, a UVB absorbing organic sunscreen), octyl methoxycinnamate (7.5% or less, a UVB absorbing organic sunscreen), octyl salicylate (also called 2-ethylhexyl salicylate; 5% or less, a UVB absorbing organic sunscreen), oxybenzone (also called benzophenone-3; 6% or less, a UVB and UVA II absorbing organic sunscreen), padimate 0 (also called octyl dimethyl PABA; 8% or less, a UVB absorbing organic sunscreen), phenylbenzimidazole sulfonic acid (water soluble; 4% or less, a UVB absorbing organic sunscreen), sulisobenzone (also called benzophenone-4; 10% or less, a UVB and UVA II absorbing organic sunscreen), titanium dioxide (25% or less, an inorganic physical blocker of UVA and UVB), trolamine salicylate (also called triethanolamine salicylate; 12% or less, a UVB absorbing organic sunscreen), and zinc oxide (25% or less, an inorganic physical blocker of UVA and UVB).

In certain embodiments, any composition or formulation described herein further comprises any suitable chelating agents, which include but are not limited to EDTA (acid form), citric acid, hydroxyethylidene phosphonic acid, polyvinylphosphoric acid, polyvinylsulfonate, acrylic acid, phytic acid, sodium phytate, and aminophosphonic acid.

In certain embodiments, any composition or formulation described herein further comprises one or more essential oil. In specific embodiments, essential oils that are optionally used in the present compositions and formulations include, but are not limited to cinnamon oil, clove oil, eucalyptus oil, garlic oil, oregano oil, jojoba oil, lavender oil, leleshwa oil, lemon oil, lemon tea tree oil, lemon myrtle oil, mint oil, neem oil, nigella sativa oil, onion oil, peppermint oil, sandalwood oil, sideritis or greek mountain tea oil, tea tree oil, thyme oil, lemongrass oil, cedarwood oil, sage oil, vetiver oil, bay oil and any combinations thereof. In some embodiments, the essential oil has antimicrobial activity.

### Methods

Provided herein in some embodiments is a method of reducing the population of pathogenic microorganisms or killing at least one pathogenic microorganism. In some embodiments, the compositions and formulations described herein are applied to skin surface. In certain embodiments, the skin surface comprising the compositions described herein is further covered with a physical barrier (e.g., dressing, medical covering for a wound, bandage, gauzes, cloth, gloves, and plastic barrier coverings). In certain embodiments, the skin surface is substantially free of microorganisms for at least 0.5 h, 1 h, 2 h, 3 h, 4 h, 5 h, 6 h, 12 h, 24 h, 36 h, 48 h, 2 days, 3 days, 4 days, 5 days, 6 days, 1 week, 2 weeks, 3 weeks, or 1 month. In other embodiments, the skin surface is substantially free of microorganisms from about 1 h to about 1 month, about 1 h to about 1 week, about 1 h to about 3 days, about 1 h to about 2 days, about 1 h to about 1 day, about 1 h to about 12 h, about 1 h to about 6 h, about 1 h to about 3 h, 6 h to about 1 month, about 6 h to about 1 week, about 6 h to about 3 days, about 6 h to about 2 days, about 6 h to about 1 day, about 6 h to about 12 h, about 12 h to about 1 month, about 12 h to about 1 week, about 12 h to about 3 days, about 12 h to about 2 days, about 12 h to about 1 day, about 24 h to about 1 month, about 24 h to about 1 week, about 24 h to about 3 days, about 24 h to about 2 days, or about 24 h to about 36 h. In certain embodiments, the skin surface is substantially free of microorganisms from about 12 h to about 24 h. In certain embodiments, the skin surface is substantially free of microorganisms from about 12 h to about 24 h when the skin surface is further covered with a physical barrier.

Provided herein in other embodiments is a method of reducing the population of pathogenic microorganisms or killing at least one pathogenic microorganism on an article surface. In some instances, the surface is hard, soft, smooth, non-porous, or porous. Examples of article surfaces include but are not limited to glass, ceramic, metal, plastic, paper, silicate, polymer or polymer/wood composites. In some embodiments, the surface is porous. In further or alternative embodiments, the surface is non-porous. Examples of porous surfaces include but are not limited to a mat of fibers, a zeolite, or a porous film. In certain embodiments, the antimicrobial composition slowly leaches from the formulation, keeping the coated surface free of live bacteria, yeasts, and molds. In further or additional embodiments, application of the composition to a surface, followed by exposure of the surface to visible or ultra-violet light, causes the destruction or inactivation of microbes or viruses that are present on the surface.

For example, in certain embodiments, a composition is present on a surface that is exposed to microbes, such as bacteria and fungi, and/or to viruses. Such a surface is a “disinfecting surface” by destroying or inactivating microbes or viruses that are present on the surface. For example, surfaces in residential, commercial or hospital environments may have a coating of an antimicrobial composition on the surface. Non-limiting examples of surfaces that may be made into disinfecting surfaces include countertops, flooring, walls, handles, telephones, and surfaces of medical instruments or devices.

In certain embodiments, the surface is a physical barrier used to cover skin surface (e.g., dressing, medical covering for a wound, bandage, gauzes, cloth, gloves, and plastic barrier coverings). In other embodiments, the surface is a medical device. In further or additional embodiments, examples of suitable medical devices include but are not limited to catheters (e.g., IV, Foley), heart valves, pacemakers, stents, gastrostomy tubes, feeding tubes, silicone coated latex type surfaces, silicone valves, balloons, septa, silicone parts used in various medical pumps, tubings, and earplugs, and as a textile finish for linings for hospital beds, window shades, and curtains.

In certain embodiments, the article surface is substantially free of microorganisms for at least 0.5 h, 1 h, 2 h, 3 h, 4 h, 5 h, 6 h, 12 h, 24 h, 36 h, 48 h, 2 days, 3 days, 4 days, 5 days, 6 days, 1 week, 2 weeks, 3 weeks, 1 month, 3 months, 6 months, 9 months, 1 year, 1.5 years, 2 years, or 5 years. In other embodiments, the article surface is substantially free of microorganisms from about 1 h to about 1 month, about 1 h to about 1 week, about 1 h to about 3 days, about 1 h to about 2 days, about 1 h to about 1 day, about 1 h to about 12 h, about 1 h to about 6 h, about 1 h to about 3 h, 6 h to about 1 month, about 6 h to about 1 week, about 6 h to about 3 days, about 6 h to about 2 days, about 6 h to about 1 day, about 6 h to about 12 h, about 12 h to about 1 month, about 12 h to about 1 week, about 12 h to about 3 days, about 12 h to about 2 days, about 12 h to about 1 day, about 24 h to about 1 month, about 24 h to about 1 week, about 24 h to about 3 days, about 24 h to about 2 days, about 24 h to about 36 h, about 1 week to about 5 years, about 1 week to about 2 years, about 1 week to about 1 year, about 1 week to about 6 months, about 1 week to about 3 months, about 1 week to about 1 month, about 1 month to about 2 years, about 1 month to about 1 year, about 1 month to about 9 months, about 1 month to about 6 months, about 1 month to about 3 months, or 1 month to about 2 months.

In some embodiments, the pathogenic microorganism is selected from, by way of non-limiting example, fungi, bacteria, viruses, protozoa, Gram-positive bacteria (e.g., Staphylococcus species, Streptococcus species, Bacillus species, and Clostridium species), Gram-negative bacteria (e.g., Escherichia species, Salmonella species, Aeromonas species, Klebsiella species and Campylobacter species), and combinations thereof. In specific embodiments, the pathogenic microorganism is selected from, by way of non-limiting example, Aspergillus niger, Pseudomonas aeruginosa, Staphylococcus aureus (MRSA), Clostridium difficile, carbapenem resistant Klebsiella pneumoniae and vancomycin-resistant Enterococci, influenza virus, H1N1 influenza virus, hepatitis A virus, hepatitis B virus, hepatitis C virus, HIV, rubella virus, human respiratory syncytial virus, mumps virus, Epstein-Barr virus, varicella zoster virus, measles virus (morbillivirus), and combinations thereof.

Provided herein, in some embodiments, is a formulation comprising nanoscale particles (e.g., nanoscale colloidal silver), wherein the release of ions (e.g., silver ions) followed by increased membrane permeability, loss of the proton motive force, inducing de-energization of the cells and efflux of phosphate, leakage of cellular content, disruption of DNA replication or combinations thereof leads to anti-bacterial or anti-microbial activity.

In some instances, the uptake of metal ions and the interactions with DNA and proteins within the bacteria (e.g., Gram-positive and Gram-negative bacteria) provides anti-bacterial or biocidal activity. In some instances, the metal ions bind with phosphate groups on DNA chains to block transcription or causes detrimental mutations. In other instances, the metal ions optionally bind to thiol groups on proteins that regulate respiration within the cell interferes with these processes, leading to cell death. In some instances, silver and other heavy metal ions optionally catalyze the production of reactive oxygen species beyond concentrations that the cells can control, leading to attacks on cell membranes and DNA damage.

In some instances, direct interactions between metal nanoscale particles and the cell wall of a Gram-negative bacterium leads to anchoring of the particle onto the cell wall or uptake of the particle into the interior of the cell. In some embodiments, these interactions lead to cell death. In some instances, shape-dependent interactions affect biocidal activity. In some instances, the shape of the nanoscale particle increases the disruptive effects of the nanoscale particles binding to bacteria cell wall.

### Formulation

Certain embodiments described herein provide the formulation as an oral care product, over-the-counter drug, over-the-counter pharmaceutical, suncare product, sunscreen product, foot-care product, liquid and bar soap, cleaning product, self-cleaning product, sanitizing product, antiperspirant product, deodorant product, fragrance product, insect repellant, cosmetic product, hair care product, shampoo, hair conditioner, hair spray, moisturizers or combinations thereof. In some embodiments, the compositions are in the form of tablets, capsules, skin patches, inhalers, eye drops, nose drops, ear drops, suppositories, creams, ointments, injectables. In certain embodiments, the product form of the present compositions are in the form of an aerosol, cream, foam, emulsion, gel, liquid, lotion, mousse, patch, pomade, powder, solid, spray, stick or towelette, or any combinations thereof.

In certain embodiments, the formulations described herein provide immediate and sustained release of one or more active agent. In other embodiments, the formulations described herein provide sustained release of one or more active agent. In further or alternative embodiments, the formulations described herein provide immediate release of one or more active agent.

In some embodiments, the compositions described herein constitute protection, treatment or care creams, sanitizers, milks, lotions, gels or foams for the face, for the hands, for the body and/or for the mucous membranes, or for cleansing the skin, or for disinfecting surfaces, or for cleansing surfaces. In certain embodiments, the compositions consist of solid preparations constituting soaps or cleansing bars. In some embodiments, the emulsions cover a broad range of consistencies including a thin lotion (which, in some instances, is also suitable for spray or aerosol delivery), creamy lotion, light cream, and heavy cream. Other suitable topical carriers include an anhydrous liquid solvent such as oil and alcohol; aqueous-based single phase liquid solvent (e.g. hydro-alcoholic solvent system); anhydrous solid and semisolid (such as a gel and a stick); and aqueous based gel and mousse system.

In certain embodiments, the nanoscale particle(s), film-forming polymer, and optional active agents are administered in the form of a composition suitable for pharmaceutical, cosmetic and industrial applications. The compositions disclosed herein may contain a pharmacologically, cosmetically or industrially acceptable carrier. Such carriers are compatible with skin, nails, mucous membranes, tissues, hair, and/or surfaces. In some embodiments, the compositions disclosed herein are in any form suitable for topical application, including aqueous, aqueous-alcoholic or oily solutions, lotion or serum dispersions, aqueous, suspension, solution, mixture, homogeneous phase formulation, anhydrous or oily gels, emulsions obtained by dispersion of a fatty phase in an aqueous phase (O/W or oil in water) or, conversely, (W/O or water in oil), microemulsions or alternatively microcapsules, multiple phase emulsions, microparticles or lipid vesicle dispersions of ionic and/or nonionic type. In some embodiments, the compositions disclosed herein comprise alcohol (e.g., SD Alcohol SDA 40-2 190 Proof, cetyl alcohol, etc.). In some embodiments, the compositions disclosed herein are alcohol-free. In some embodiments, the compositions disclosed herein are formulated as composite films, paints or fibers.

In other embodiments, the composition further comprises at least one of water, a preservative, a surfactant (e.g., Incromine® Oxide C), an antioxidant (vitamin E acetate), an emulsifier (e.g. Emulium® Kappa), a conditioner, an emollient, a wax (e.g., Cutina® CP), an oil, a polymer, a pH adjuster (e.g., AMP Ultra® PC 2000) an adjuvant (e.g., hydrophilic or lipophilic gelling agents), a thickener (e.g., Cosmedia® Ultragel 300, Structure® Solanace, Keltrol® xanthan gum, etc.), a fixative, a colorant, a humectant, a moisturizer, a stabilizer, a diluent, a solvent (e.g, Dermofeel® TC-7), a filler, a sunscreen, an odor-absorber, a dyestuff, and a fragrance.

In some embodiments, the compositions and formulations described herein further comprise a vehicle acceptable for topical application to the skin or hair. Examples of such vehicles include, but are not limited to, water and aqueous systems (e.g., deionized water, sterile water); glycerin; various hydrophilic solvents including alcohols such as ethanol, methanol, propyl and other alcohols; polyglycols (e.g., glycerol or polyethylene glycol), esters of fatty acids, oils, fats, silicones, and the like.

In some embodiments, any composition or formulation described herein optionally comprises at least one preservative. Suitable preservatives include, but are not limited to, acids, alcohols, glycols, parabens, sorbates (e.g., potassium sorbate), quaternary-nitrogen containing compounds, isothiazolinones, aldehyde-releasing compounds and halogenated compounds. Illustrative alcohols include phenoxyethanol, isopropyl alcohol, and benzyl alcohol; illustrative glycols include propylene, butylene and pentylene glycols (e.g., 1,3-butylene glycol); illustrative parabens include (also known as parahydroxybenzioc acids) methyl, propyl and butyl parabens; illustrative quaternary nitrogen containing compounds include benzalkonium chloride, Quartenium 15; illustrative isothiazoles include methylisothiazoline, methychlorolisothiazoline; illustrative aldehyde releasing agents include DMDM hydantion, imiadolidinyl urea and diazolidinyl urea; illustrative antioxidants include butylated hydroxytoluene, tocopherol and illustrative halogenated compounds include triclosan and chlorohexidene digluconate. In some embodiments, any of the compositions described herein optionally comprise Euxyl® PE 9010. In some embodiments, any of the compositions described herein optionally comprise cocamidopropyl PC-dimonium chloride phosphate (e.g., Arlasilk® PTC). Examples of preservatives useful for the purpose of the present disclosure can be found in Steinberg, D. “Frequency of Use of Preservatives 2007”. Cosmet. Toilet. 117, 41-44 (2002) and, “Preservative Encyclopedia” Cosmet. Toilet. 117, 80-96 (2002). In addition, enzyme preservative systems such as those described in the article by Ciccognani D. Cosmetic Preservation Using Enzymes, in “Cosmetic and Drug Microbiology”, Orth DS ed., Francis & Taylor, Boca Raton, Fla. (2006) can also be effective for use with the composition of the present disclosure.

Compositions disclosed herein are formulated in conventional manner using one or more pharmaceutically, cosmetically, or industrially acceptable carriers comprising excipients and auxiliaries which facilitate processing of the nanoscale particle(s), film-forming polymer, and optional agents. Proper formulation is dependent upon the route of administration chosen and standard therapeutic practice. As used herein, the term “pharmaceutically, cosmetically or industrially acceptable carrier” means an inert, non toxic solid or liquid filler, diluent or encapsulating material, not reacting adversely with the active compound or with the subject. Suitable carriers include water, saline, aqueous dextrose, sugar solutions, ethanol, glycols and oils, including those of petroleum, animal, vegetable, or synthetic origin, for example, peanut oil, soybean oil and mineral oil. In other embodiments, an active agent or combination of active agents described herein is optionally formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate, sodium lauryl sulfate and BRIJ® IC20-70), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Some embodiments provided herein describe a sprayable formulation comprising nanoscale particles (e.g., silver nanocrystals). In some embodiments, the formulation comprises a bimodal distribution of particle sizes, wherein the smaller particles provide immediate and high ion release rates to kill microbes (e.g., bacteria) on surfaces and the larger particles provide a long-term ion source. In other embodiments, the formulation comprises a polydisperse size population of particles. In some embodiments, the formulation optionally comprises a biocompatible polymer. In some embodiments, one or more polymer additives provide a thin film to adhere the formulation to surfaces. In some embodiments, the polymer film is hygroscopic. In some instances, the hygroscopic film absorbs water from the atmosphere to enhance silver oxidation and/or provides channels for ion transport.

For oral administration, the compositions, in some embodiments, take the form of, for example, tablets or capsules prepared by conventional means with acceptable excipients or carriers such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolae); or wetting agents (e.g., sodium lauryl sulphate). Liquid preparations for oral administration are, in certain embodiments, solutions, syrups or suspensions, or they are presented as a dry product for constitution with water or other suitable vehicle before use. Such liquid preparations are prepared by conventional means with acceptable excipients or carriers such as suspending agents (e.g., sorbitol syrup cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). In some embodiments, the preparations optionally contain buffer salts, flavoring, coloring and sweetening agents as appropriate.

In some embodiments, topical compositions disclosed herein are in the form of a viscous liquid, solution, suspension, liposomal formulations, gel, jelly, cream, lotion, ointment, suppository, foam, aerosol spray aqueous or oily suspensions or solutions, emulsions, or emulsion ointments. In one embodiment, a topical composition is provided which includes a topical carrier. For example, thickeners, diluents, emulsifiers, dispersing aids or binders are optionally used as needed. The topical carrier is selected so as to provide the composition in the desired form, e.g., as a liquid, lotion, cream, paste, gel, powder, or ointment, and are comprised of a material of either naturally occurring or synthetic origin. Examples of suitable topical carriers for use herein include water, alcohols and other nontoxic organic solvents, glycerin, mineral oil, silicone, petroleum jelly, lanolin, fatty acids, vegetable oils, parabens, aloe vera, waxes, and the like. In some embodiments, topical formulations for application to skin include ointments, lotions, pastes, creams, gels, drops, suppositories, sprays, liquids, powders, shampoos, and transdermal patches.

In certain embodiments, ointments and creams are, for example, formulated with an aqueous or oily base with the addition of suitable thickening and/or gelling agents. Lotions are formulated with an aqueous or oily base and will in general also containing one or more emulsifying agents, stabilizing agents, dispersing agents, suspending agents, thickening agents, or coloring agents.

Any composition described herein optionally comprises one or more lubricant(s) such as, but not limited to, calcium tearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, Dow Corning 200® Fluid 100 cs, a syloid silica gel, a coagulated aerosol of synthetic silica, or mixtures thereof.

In some embodiments, one function of the carrier is to enhance surface penetration of the active ingredients. Suitable carriers include liposomes, ethanol, dimethylsulfoxide (DMSO), petroleum jelly (petrolatum), mineral oil (liquid petrolatum), water, dimethylformamide, dekaoxyethylene-oleylether, oleic acid, 2-pyrrolidone, and Azone® brand penetration enhancer (Upjohn).

In some embodiments, one or more nanoparticles are encapsulated in a liposome. In further or additional embodiments, a composition described herein comprises one or more nanoparticles encapsulated in a liposome and a moisturizing agent. In certain embodiments, the encapsulated nanoparticle provides for sustained release of the nanoparticle.

In one embodiment, the compositions are in a form suitable for cosmetic application including, but not limited to, lotions, ointments, creams, sprays, spritzes, aqueous or aqueous-alcoholic mixture gels, mousses, patches, pads, masks, moistened clothes, wipes, solid sticks, clear sticks, lip sticks, aerosol creams, anhydrous powders, talcs, tonics, oils, emulsions or bath salts.

In another embodiment, the composition optionally contains irritation-mitigating additives to minimize or eliminate the possibility of skin irritation or skin damage resulting from the chemical compound to be administered, or other components of the composition. Suitable irritation-mitigating additives include for example: α-tocopherol, monoamine oxidase inhibitors (e.g., 2-phenyl-1-ethanol), glycerin, salicylates, ascorbates, ionophores (e.g., monensin), amphiphilic amines, avenanthramides (e.g., SymCalmin® 143535), DragoCalm®, ammonium chloride, N-acetylcysteine, capsaicin, and/or chloroquine.

The “effective amount”, however, will take into account any toxicity effects that may occur, for example, severe skin irritation with higher doses of the active agents disclosed herein. Suggested endpoints may first be measured in vitro or in an animal model to determine the acceptable range of active agents to be used in conjunction with the compositions disclosed herein. The “effective amount” varies depending on the compound, the disease and its severity, and the age, weight, etc., of the subject to be treated.

In certain embodiments, the compositions and formulations disclosed herein comprises a nanoscale particle in a concentration of about 0.0000001%, about 0.0000005%, about 0.000001%, about 0.000002%, about 0.000004%, about 0.000006%, about 0.000008%, about 0.00001%, about 0.0001% about 0.001%, about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprises a nanoscale particle in a concentration from about 0.0000001% to about 5%, from about 0.0000001% to about 1%, from about 0.000001% to about 1%, from about 0.0000001% to about 0.001%, from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprises nanoscale titanium dioxide in a concentration of about 0.005%, about 0.008%, about 0.01%, about 0.05%, about 0.08%, about 0.1%, about 0.15%, about 0.175%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprises nanoscale titanium dioxide in a concentration from about 0.005% to about 50%, from about 0.05% to about 5%, from about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprises nanoscale titanium dioxide in an amount of about 0.01 mg/mL to about 3 mg/mL, about 0.02 mg/mL to about 3 mg/mL, about 0.03 mg/mL to about 3 mg/mL, about 0.04 mg/mL to about 3 mg/mL, about 0.05 mg/mL to about 3 mg/mL, about 0.06 mg/mL to about 3 mg/mL, about 0.07 mg/mL to about 3 mg/mL, about 0.08 mg/mL to about 3 mg/mL, about 0.09 mg/mL to about 3 mg/mL, about 0.1 mg/mL to about 3 mg/mL, about 0.01 mg/mL to about 2.9 mg/mL, about 0.01 mg/mL to about 2.8 mg/mL, about 0.01 mg/mL to about 2.6 mg/mL, about 0.01 mg/mL to about 2.5 mg/mL, about 0.01 mg/mL to about 2.4 mg/mL, about 0.01 mg/mL to about 2.3 mg/mL, about 0.01 mg/mL to about 2.2 mg/mL, about 0.01 mg/mL to about 2.1 mg/mL, about 0.01 mg/mL to about 2 mg/mL, about 0.01 mg/mL to about 1.9 mg/mL, about 0.01 mg/mL to about 1.8 mg/mL, about 0.01 mg/mL to about 1.7 mg/mL, about 0.1 mg/mL to about 2 mg/mL, about 0.5 mg/mL to about 2 mg/mL, about 1 mg/mL to about 3 mg/mL, about 1.5 mg/mL to about 1.9 mg/mL, about 0.1 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, or about 3 mg/mL.

In certain embodiments, the compositions and formulations disclosed herein comprises nanoscale titanium in a concentration of about 0.005%, about 0.008%, about 0.01%, about 0.05%, about 0.08%, about 0.1%, about 0.15%, about 0.175%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprises nanoscale titanium in a concentration from about 0.005% to about 50%, from about 0.05% to about 5%, from about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, from about 0.05% to about 0.2%, or from about 0.005% to about 2% by weight relative to the total weight of the composition or formulation. In specific embodiments, the compositions and formulations disclosed herein comprises nanoscale titanium in a concentration of about 0.05%, 0.075%, 0.1%, 0.125%, 0.15%, 0.175%, 0.2%, 0.225%, 0.25%, 0.275%, or 0.3%.

In certain embodiments, the compositions and formulations disclosed herein comprises nanoscale titanium in an amount of about 0.01 mg/mL to about 3 mg/mL, about 0.02 mg/mL to about 3 mg/mL, about 0.03 mg/mL to about 3 mg/mL, about 0.04 mg/mL to about 3 mg/mL, about 0.05 mg/mL to about 3 mg/mL, about 0.06 mg/mL to about 3 mg/mL, about 0.07 mg/mL to about 3 mg/mL, about 0.08 mg/mL to about 3 mg/mL, about 0.09 mg/mL to about 3 mg/mL, about 0.1 mg/mL to about 3 mg/mL, about 0.01 mg/mL to about 2.9 mg/mL, about 0.01 mg/mL to about 2.8 mg/mL, about 0.01 mg/mL to about 2.6 mg/mL, about 0.01 mg/mL to about 2.5 mg/mL, about 0.01 mg/mL to about 2.4 mg/mL, about 0.01 mg/mL to about 2.3 mg/mL, about 0.01 mg/mL to about 2.2 mg/mL, about 0.01 mg/mL to about 2.1 mg/mL, about 0.01 mg/mL to about 2 mg/mL, about 0.01 mg/mL to about 1.9 mg/mL, about 0.01 mg/mL to about 1.8 mg/mL, about 0.01 mg/mL to about 1.7 mg/mL, about 0.1 mg/mL to about 2 mg/mL, about 0.5 mg/mL to about 2 mg/mL, about 1 mg/mL to about 3 mg/mL, about 1.5 mg/mL to about 1.9 mg/mL, about 0.1 mg/mL, about 0.25 mg/mL, about 0.5 mg/mL, about 0.75 mg/mL, about 1 mg/mL, about 1.25 mg/mL, about 1.5 mg/mL, about 1.75 mg/mL, about 2 mg/mL, about 2.25 mg/mL, about 2.5 mg/mL, about 2.75 mg/mL, or about 3 mg/mL.

In certain embodiments, the compositions and formulations disclosed herein comprises nanoscale colloidal silver in a concentration of about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprises nanoscale colloidal silver in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprises nanoscale silver salt in a concentration of about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprises nanoscale silver salt in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In certain embodiments, the compositions and formulations disclosed herein comprises silver in a concentration of about 0.0000001%, about 0.0000002%, about 0.0000004%, about 0.0000006%, about 0.0000008%, about 0.000001%, about 0.000002%, about 0.000004%, about 0.000006%, about 0.000008%, about 0.00001%, about 0.00002%, about 0.00004%, about 0.00006%, about 0.00008%, about 0.0001%, about 0.0002%, about 0.0004%, about 0.0006%, about 0.0008%, about 0.001%, about 0.002%, about 0.004%, about 0.006%, about 0.008%, about 0.01%, about 0.02%, about 0.04%, about 0.06%, about 0.08%, or about 0.1%, by weight relative to the total weight of the composition or formulation. Preferably, the compositions and formulations disclosed herein comprises silver in a concentration from about 0.0000001% to about 5%, from about 0.0000001% to about 1%, from about 0.000001% to about 1%, from about 0.0000001% to about 0.01%, from about 0.0000001% to about 0.001%, from about 0.0000002% to about 0.001%, from about 0.0000004% to about 0.001%, from about 0.0000006% to about 0.001%, from about 0.0000008% to about 0.001%, from about 0.000001% to about 0.001%, from about 0.000002% to about 0.001%, from about 0.000004% to about 0.001%, from about 0.000006% to about 0.001%, from about 0.000008% to about 0.0001%, or from about 0.000008% to about 0.0001% by weight relative to the total weight of the composition or formulation.

In certain embodiments, the compositions and formulations disclosed herein comprises silver in an amount of about 0.001 mg/mL to about 0.1 mg/mL, about 0.002 mg/mL to about 0.1 mg/mL, about 0.004 mg/mL to about 0.1 mg/mL, about 0.006 mg/mL to about 0.1 mg/mL, about 0.008 mg/mL to about 0.1 mg/mL, about 0.01 mg/mL to about 0.1 mg/mL, about 0.02 mg/mL to about 0.1 mg/mL, about 0.04 mg/mL to about 0.1 mg/mL, about 0.06 mg/mL to about 0.1 mg/mL, about 0.001 mg/mL to about 0.08 mg/mL, about 0.001 mg/mL to about 0.06 mg/mL, about 0.001 mg/mL, about 0.002 mg/mL, about 0.003 mg/mL, about 0.004 mg/mL, about 0.005 mg/mL, about 0.006 mg/mL, about 0.007 mg/mL, about 0.008 mg/mL, about 0.009 mg/mL, about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, or about 0.15 mg/mL.

In certain embodiments, the compositions and formulations disclosed herein comprises nanoscale zinc oxide in a concentration of about 0.00005%, about 0.005%, about 0.008%, about 0.01%, about 0.05%, about 0.08%, about 0.1%, about 0.15%, about 0.2%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation. Preferably, the compositions and formulations disclosed herein comprises nanoscale zinc oxide in a concentration from about 0.005% to about 50%, about 0.01% to about 50%, from about 0.1% to about 30%, from about 0.1% to about 20%, from about 0.5% to about 20%, from about 0.5% to about 10%, from about 0.5% to about 5%, from about 0.5% to about 3%, from about 0.5% to about 2.0%, from about 0.5% to about 1.5%, from about 0.75% to about 10%, from about 0.75% to about 7.5%, from about 0.75% to about 5%, from about 1% to about 10%, from about 1% to about 5%, from about 1% to about 2.5%, from about 1% to about 2%, from about 0.5% to about 2%, or from about 0.005% to about 2% by weight relative to the total weight of the composition or formulation.

In some embodiments, the compositions and formulations described herein comprise a film-forming polymer in an amount from about 0.05% to 50%, from about 0.05% to about 25%, from about 0.05% to about 20%, from about 0.05% to about 15%, from about 0.05% to about 10%, from about 0.05% to about 5%, from about 0.05% to about 2%, from about 0.05% to about 1%, from about 0.1% to about 10%, from about 0.1% to about 5%, from about 0.1% to about 3%, from about 0.1% to about 2%, from about 0.1% to about 1%, from about 1% to about 20%, from about 1% to about 30%, from about 1% to about 40%, from about 1% to about 50% by weight relative to the total weight of the composition. In some embodiments, the compositions and formulations described herein comprise a film-forming polymer in an amount in an amount of about 0.005%, about 0.008%, about 0.01%, about 0.05%, about 0.1%, about 0.2%, about 0.25%, about 0.3%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise polyolprepolymer-2 in an amount from about 0.05% to 50%, from about 0.05% to about 25%, from about 0.05% to about 20%, from about 0.05% to about 15%, from about 0.05% to about 10%, from about 0.05% to about 5%, from about 0.05% to about 2%, from about 0.05% to about 1%, from about 0.1% to about 10%, from about 0.1% to about 5%, from about 0.1% to about 3%, from about 0.1% to about 2%, from about 0.1% to about 1%, from about 1% to about 20%, from about 1% to about 30%, from about 1% to about 40%, from about 1% to about 50% by weight relative to the total weight of the composition. In some embodiments, the compositions and formulations described herein comprise polyolprepolymer-2 in an amount in an amount of about 0.005%, about 0.008%, about 0.01%, about 0.05%, about 0.1%, about 0.2%, about 0.25%, about 0.3%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise an iodine source in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise an iodine source in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise povidone-iodine in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise povidone-iodine in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise a surfactant in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise a surfactant in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In some embodiments, the compositions and formulations described herein comprise benzalkonium chloride in an amount from about 0.05% to 50%, from about 0.05% to about 25%, from about 0.05% to about 20%, from about 0.05% to about 15%, from about 0.05% to about 10%, from about 1% to about 20%, from about 1% to about 30%, from about 1% to about 40%, from about 1% to about 50% by weight relative to the total weight of the composition. In some embodiments, the compositions and formulations described herein comprise benzalkonium chloride in an amount in an amount of about 0.005%, about 0.008%, about 0.01%, about 0.05%, about 0.1%, about 0.5%, about 0.8%, about 1%, about 1.1%, about 1.2%, about 1.3%, about 1.4%, about 1.5%, about 1.6%, about 1.7%, about 1.8%, about 1.9%, about 2%, about 2.3%, about 2.5%, about 2.8%, about 3%, about 3.3%, about 3.5%, about 3.8%, about 4%, about 4.3%, about 4.5%, about 4.7%, about 5%, about 5.3%, about 5.5%, about 5.7%, about 6%, about 6.5%, about 7%, about 8%, about 10%, about 13%, about 15%, about 18%, about 20%, about 22% or about 25% by weight relative to the total weight of the composition or formulation.

In certain embodiments, the compositions and formulations disclosed herein comprises a moisturizer in a concentration of about 0.5% to about 15%, about 5% to about 15%, about 10% to about 25%, about 10% to about 50%, about 10% to about 75%, about 10% to about 95%, about 0.5% to about 95%, about 5% to about 75%, about 15% to about 75%, about 25% to about 75%, about 50% to about 75%, about 15% to about 25%, about 15% to about 50%, about greater than 1%, greater than 5%, greater than 10%, greater than 20%, greater than 50%, less than about 90%, less than about 80%, less than about 70%, less than about 60%, less than about 50%, less than about 40%, less than about 30%, less than about 25%, less than about 20%, less than about 15%, less than about 10%, less than about 5%, about 0.05%, about 0.1%, about 0.5%, about 1% about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, about 95%, or about 98% by weight relative to the total weight of the composition or formulation.

### Administration

Certain embodiments describe a composition that is applied to a surface to provide a temporary and/or sustained disinfection of the surface.

Examples of routes of administration to a mammal include, but are not limited to, oral, buccal, inhalation, intradermal, subcutaneous, transmucosal, transdermal, or topical administration. Topical administration may also involve the use of transdermal administration such as transdermal patches or iontophoresis devices. Certain transdermal patches for the delivery of pharmaceutical agents are known. See, e.g., U.S. Pat. Nos. 5,023,252, 4,992,445, and 5,001,139. Such patches are be constructed for continuous, pulsatile, or on demand delivery of pharmaceutical agents.

In some embodiments, any of the compositions and formulations described herein are used in combination with another agent. In some embodiments, a composition described herein and an additional agent are administered to a surface simultaneously. In other embodiments, a composition described herein and an additional agent are administered at staggered times. In some embodiments, a composition described herein and an additional agent are mixed together prior to application.

In some embodiments, any of the composition and formulations described herein are heated prior to administration to a surface. In other embodiments, the compositions and formulations described herein are heated after administration to a surface. In some embodiments, the compositions and formulations described herein are heated to 40° C., 50° C., 60° C., 70° C., 80° C., 90° C., 100° C., 125° C., 150° C., 175° C., or 200° C. In some instances, heating the composition or formulation enhances the antimicrobial activity.

In some embodiments, any of the compositions or formulations described herein are applied as needed or alternatively as a part of a disinfecting routine. In some embodiments, the composition is applied to a surface once, twice or three times daily. In other embodiments, the composition is applied to a surface once weekly, monthly, or yearly. In additional or further embodiments, the composition is applied to a surface 2, 3, 4, or 5 times weekly. In certain embodiments, the composition is applied to a surface every 3 hours, 6 hours, 12 hours, 24 hours, 36 hours, 48 hours, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years.

### EXAMPLES

### Example 1

### Nanoscale Particle Antimicrobial Composition

Exemplary antimicrobial compositions (on a weight percent basis, based on the total weight of the composition) are given below with water/aqua/eau constituting the balance of the composition.

| | Ranges % | A % | B % | C % | D % |
|---|---|---|---|---|---|
| Component | (w/w) | (w/w) | (w/w) | (w/w) | (w/w) |
| Ethyl Alcohol | 40-75 | 62 | 58 | 60 | 55 |
| Polyquaternium-37 | 1-6 | 3 | 3 | 2.8 | 2.5 |
| PPG-12/SMDI | 0.01-5 | 0.75 | 0.01 | 1.1 | 0.75 |
| Copolymer | | | | | |
| Pentylene Glycol | 0.001-3.5 | 0.5 | 0.5 | 0.6 | 0.5 |
| Butylene Glycol | 0.001-3.5 | 0.5 | 0.5 | 0.5 | 0.5 |
| Leptospermum | 0.001-3 | 0.15 | 0.2 | 0.15 | 0.18 |
| Petersonii Oil | | | | | |
| Cetrimonium Chloride | 0.005-2 | 0.3 | 0.3 | 0.3 | 0.3 |
| Titanium Dioxide | 0.005-1.75 | 0.2 | — | 0.25 | 0.2 |
| Nanoparticle | | | | | |
| Benzalkonium Chloride | 0.005-2.5 | 0.2 | 0.2 | 0.2 | 0.5 |
| Hydroxyphenyl | 0.005-2.5 | 0.5 | 0.5 | 0.5 | 0.5 |
| Propamidobenzoic | | | | | |
| Acid | | | | | |
| Yellow 5 | 0.001-0.1 | 0.05 | 0.04 | 0.03 | 0.03 |
| Colloidal Silver Source | 0.01-1.5 | 0.1 | 0.25 | — | — |
| Base Product | | | | | |
| Silver citrate | 0.5-30 | — | — | — | 10 |
| Silver-copper alloy | 0.05-15 | — | — | — | 5 |
| nanopowder | | | | | |
| Citric acid | 0.005-20 | — | — | — | 10 |

### Example 2

### Nanoscale Particle Antimicrobial Composition

Exemplary antimicrobial compositions (on a weight percent basis, based on the total weight of the composition) are given below.

| | Ranges % | A % | B % | C % | D % |
|---|---|---|---|---|---|
| Component | (w/w) | (w/w) | (w/w) | (w/w) | (w/w) |
| Deionized Water | 15-85% | 20.14 | 30.33 | 82.61 | 82.5 |
| Yellow 5 - 1% | | 0.95 | 0.90 | 0.95 | 0.90 |
| solution* | | | | | |
| Elemental silver | 3.8-4.8 | 4 | 4.5 | 4 | 4.8 |
| nanoparticle (100 nm) | | | | | |
| Titanium Dioxide | 0-6 | — | — | 5 | 4.3 |
| nanoparticle | | | | | |
| Titanium dioxide USP | 2-3 | 2.5 | 2.7 | 2.5 | 2.4 |
| #3328 | | | | | |
| SD Alcohol SDA 40-2 | 0-70 | 67.2 | 57.01 | — | — |
| (190 Proof) | | | | | |
| SymCalmin ® No. | 0.5-1.5 | 1 | 1.06 | 1 | 0.8 |
| 143535 | | | | | |
| Cosmedia ® Ultragel | 0.5-2.5 | 2 | 1.5 | 0.95 | 1.2 |
| 300 | | | | | |
| Euxyl ® PE 9010 | 0-2 | — | — | 1 | 1.3 |
| PPG-12/SMDI | 1.5-2.5 | 1.75 | 1.8 | 1.75 | 1.5 |
| Copolymer | | | | | |
| Stepanquat ® 50 NF | 0-0.4 | 0.26 | 0.1 | — | — |
| Lemon Tea Tree Oil | 0.1-0.3 | 0.2 | 0.1 | 0.2 | 0.25 |
| AMP ® Ultra PC 2000 | 0.01-0.07 | _{—} | — | 0.04 | 0.05 |

*1% solution comprises 89% deionized water, 10% incroquat, 1% FD&C Yellow 5

### Example 3

### Nanoscale Particle Antimicrobial Moisturizer

Exemplary antimicrobial moisturizers (on a weight percent basis, based on the total weight of the composition) are given below.

| | Ranges % | A % | B % | C % | D % |
|---|---|---|---|---|---|
| Component | (w/w) | (w/w) | (w/w) | (w/w) | (w/w) |
| Deionized Water | 55-65 | 61.14 | 61.57 | 59.77 | 61.05 |
| Structure ® Solanace | 0.5-1.5 | 1 | 0.9 | 1.2 | 0.8 |
| 28-1808 | | | | | |
| Elemental silver | 3.5-4.5 | 4 | 4.2 | 3.8 | 4.1 |
| nanoparticle (100 nm) | | | | | |
| Keltrol CG-RD | 0.15-0.25 | 0.2 | 0.2 | 0.18 | 0.2 |
| Glycerin USP 99.7% | 2-4 | 3 | 2.8 | 3 | 3.2 |
| D-Panthenol 75L | 0.5-2 | 1 | 0.9 | 0.8 | 1 |
| Potassium Sorbate PDR | 0.05-0.3 | 0.1 | 0.1 | 0.2 | 0.15 |
| FCC K | | | | | |
| Euxyl ® PE 9010 | 0.5-1.5 | 1 | 1 | 1.1 | 0.8 |
| Emulium ® Kappa | 3.8-4.8 | 4 | 4.2 | 4.4 | 4 |
| Cetyl Alcohol C16-98, | 2-3 | 2.5 | 2.6 | 2.4 | 2.5 |
| NF Grade | | | | | |
| Cutina ® CP | 1.8-2.5 | 2 | 2 | 2.3 | 2.2 |
| Dermofeel ® TC-7 | 3.5-6 | 5 | 4 | 5 | 4.5 |
| Hydromide ® Blend | 1.5-2.5 | 2 | 2 | 2.1 | 2.1 |
| Net Sterol-100 | 0.25-1 | 0.5 | 0.5 | 0.5 | 0.6 |
| Jojoba Oil Golden | 0.5-1.8 | 1 | 1.5 | 0.9 | 1 |
| High Oleic Sunflower | 1-3 | 2 | 1.5 | 1.8 | 2 |
| Oil | | | | | |
| Dow Corning 200 ® | 1-3 | 2 | 2.1 | 2.3 | 1.8 |
| Fluid 100 cs | | | | | |
| Vitamin E Acetate | 0.05-0.3 | 0.1 | 0.1 | 0.1 | 0.2 |
| USP/FCC | | | | | |
| Liponate ® GC | 3-4.5 | 3.5 | 3.5 | 3.7 | 3.5 |
| DragoCalm ® | 1.5-3 | 2 | 2.3 | 2.4 | 2.1 |
| PPG-12/SMDI | 1-3 | 1.5 | 1.6 | 1.5 | 1.8 |
| Copolymer | | | | | |
| Stepanquat ® 50 NF | 0.1-0.5 | 0.26 | 0.25 | 0.3 | 0.2 |
| Lavender Royale “B” | 0.1-0.4 | 0.2 | 0.18 | 0.25 | 0.2 |

### Example 4

### Nanoscale Particle Antimicrobial Foam Soap

Exemplary antimicrobial compositions (on a weight percent basis, based on the total weight of the composition) are given below.

| | Ranges % | A % | B % | C % | D % |
|---|---|---|---|---|---|
| Component | (w/w) | (w/w) | (w/w) | (w/w) | (w/w) |
| Deionized Water | 70-80 | 76.390 | 78.5 | 76.32 | 75.75 |
| 1,3-Butylene glycol | 2.2-3.5 | 3 | 2.5 | 2.8 | 2.6 |
| BRIJ ® IC20-70 | 0.5-2 | 1.25 | 1 | 1.3 | 1.5 |
| Euxyl ® PE 9010 | 0.3-2 | 1 | 1 | 1.3 | 0.9 |
| Lemon Tea Tree Oil | 0-0.5 | 0.1 | 0.15 | 0.1 | 0.1 |
| Incromine ® Oxide C | 8-14 | 12 | 10 | 12 | 13 |
| Arlasilk ® PTC | 1.4-2.5 | 2 | 2.3 | 2 | 1.8 |
| Stepanquat ® 50 NF | 0.1-0.3 | 0.26 | 0.25 | 0.28 | 0.25 |
| Elemental silver | 3.5-4.8 | 4 | 4.3 | 3.9 | 4.1 |
| nanoparticle (100 nm) | | | | | |

### Example 5

### Stability of Nanoscale Particle Antimicrobial Compositions

Compositions 1A-D, 2A-D, 3A-D, and 4A-D are monitored over time to assess the stability of the formulations against nanoparticle aggregation. Stability is measured using optical spectroscopy to measure changes in solution turbidity over time. Dynamic light scattering (DLS) is used to measure changes in the hydrodynamic diameter of the aggregates over time, providing information on the size of the aggregates present. Turbidity and DLS measurements of the compositions are conducted at 0° C., 5° C., 15° C., 25° C., 35° C., 50° C., and 75° C.

### Example 6

### Film Morphology and Particle Distribution of Nanoscale Particle Antimicrobial Compositions

Compositions 1A-D, 2A-D, 3A-D, and 4A-D are sprayed onto glass slides positions at various locations within the spray pattern. Optical microscopy is used to examine the morphology of the polymer/nanoparticle film. Bright field measurements are used to analyze the gross morphology of the polymer film. Dark field measurements are used to measure the distribution of silver nanoparticles within the film.

### Example 7

### Ion Release Rates of Nanoscale Particle Antimicrobial Compositions

Compositions 1A-D, 2A-D, 3A-D, and 4A-D are coated on several substrates and allowed to dry to form a film. A baseline for the initial amount of silver present in the films is established with digestion of a film with nitric acid, followed by analysis of silver content using inductively coupled plasma mass spectroscopy (ICP-MS) or atomic absorption spectroscopy (AAS). The remaining films are aged, undisturbed, for varying amounts of time. At each time point (e.g., 1 h, 2 h, 3 h, 4 h, 5 h, 6 h, 12 h, 24 h, 36 h, 48 h, and 1 week), a portion of the film is dissolved into water using sonication and agitation. The resultant solution is passed through an ultrafine filer to remove undissolved nanoparticles. The solution is treated with nitric acid to separate bound silver ions from any polymer and the silver concentration is measured using ICP-MS analysis. Dissolution kinetics are obtained by analysis of the data at different time points.

### Example 8

### Disk Diffusion Assay

A panel of representative bacteria is utilized to evaluate the efficacy of the antimicrobial formulations. Cultures of Pseudomonas aerugenosa, Clostridium perfingens, Clostridium difficile and Staphylococcus aureus are obtained from American Type Culture Collection. Screening is accomplished using a disk diffusion assay to provide a qualitative means of comparing the release of silver ions and anti-bacterial properties of thin-films of nanoparticles. Plates are prepared containing a nutrient agar for the growth of the bacteria, followed by the uniform application of bacterial culture. Thin film samples of Examples 1A, 2A and 2C are prepared by filtering nanoparticle solutions through filter paper, drying the filter paper, and punching small coupons from the dried paper. The mass concentration of silver in the samples is determined by digesting the silver nanoparticles from a coupon in nitric acid solution, followed by quantitative measurement of the silver ion concentration using inductively coupled plasma mass spectroscopy (ICP-MS) or atomic absorption spectroscopy (AAS). The thin film samples of Examples 1A, 2A and 2C are placed into the cultured plate. The plates are incubated at 37° C. for 24 hours. Following incubation, the samples are analyzed for inhibition zones where bacteria are killed.

### Example 9

### In Vitro Efficacy of the Nanoscale Particle Antimicrobial Composition 1A

Minimum inhibitory concentration studies are performed using the gram-negative enterobacterium Pseudomonas aerugenosa (American Type Culture Collection #9027) in accordance with the protocol for testing the bactericidal activity of antimicrobial agents (Document M26-T of the National Center for Clinical and Laboratory Standards). P. aerugenosa are cultured overnight at 37° C. in trypsin soy broth to a final density of approximately 1×10⁸ cfu/ml (0.5 McFarland standard) and then diluted 1:10 with cation-adjusted Mueller-Hinton medium. 10 microliters of this bacterial culture are added to 200 microliters of an already-prepared dilution series of the test antimicrobial solution comprising Composition 1A. After a 5 minute incubation at room temperature, 10 microliters of wash test solution are plated onto a sector of a Letheen-agar plate and incubated at 37° C. overnight. MIC breakpoint is interpreted as the highest dilution for which no growth was evident.

### Example 10

### Efficacy of the Nanoscale Particle Antimicrobial Composition 1A and 2A on Article Surface

Duplicate samples of two silicone catheters, one coated with the Nanoscale Particle Antimicrobial Composition 1A, one coated with Composition 2A and one uncoated control, are cut into 2-cm length samples and each are placed in a separate sterile tube. Inoculum cultures of 1×10⁵ E. coli cells/mL (clinical isolate from UTI) in synthetic urine are prepared. 1 mL of synthetic urine solution and 1 mL inoculum culture are added to each tube containing the catheter samples, and the tubes are incubated at 37° C., rotating at 20 rpm. (Day 0)

Upon completion of incubation time (Days 1, 4), the following assays are performed on separate duplicate samples for each day of incubation: (1) planktonic growth of the contacting solution (CS); and (2) counting of attached viable cells (biofilm) on the catheter pieces (S). The dilutions tested were: 1:10, 10², 10³, 10⁴. On day 1, the S and CS solutions are plated in parallel onto MacConkey plates.

Duplicate samples of 5 different coated catheters, and one uncoated control catheter, are cut into 2-cm length samples and each is placed in a separate sterile tube. An inoculum culture of 1×10⁵ E. coli cells/mL (clinical isolate from UTI) in synthetic urine is prepared. 1 mL of the synthetic urine solution and 1 mL of the inoculum culture are added to each tube containing the catheter samples and the tubes are incubated at 37° C., rotating at 20 rpm (Day 0). Upon completion of the incubation times (Days 1, 2, 4, and 7), the following assays are performed on separate duplicate samples for each day of incubation: (a) planktonic growth of the contacting solution (CS); and (b) counting of attached viable cells (biofilm) on the catheter pieces (S). On Day 4, only the contacting solution is assayed. The catheter samples are not sonicated, but rather are transferred to new test tubes with a fresh challenge of 1×10⁵ E. coli cells. After an additional 3 days (which corresponded to Day 7 of the overall experiment), these samples are assayed for planktonic growth of the contacting solution (CS) and for attached viable cells (biofilm) (S).

### Example 11

### Efficacy of the Nanoscale Particle Antimicrobial Compositions on Article Surfaces

Small test objects with surfaces of stainless steel, ceramic tile, glass or paint are obtained. Four sets of the test objects of each material are exposed to Pseudomonas aerugenosa, Clostridium perfingens, Clostridium difficile or Staphylococcus aureus, then sprayed with Antimicrobial Composition 1A, 1B, 1C, 1D, 2A, 2B, 2C, 2D, 3A, 3B, 3C, 3D, 4A, 4B, 4C, and/or 4D. Another four sets of test objects of each material are sprayed with the antimicrobial composition. After the composition has dried, the test objects are exposed to Pseudomonas aerugenosa, Clostridium perfingens, Clostridium difficile or Staphylococcus aureus. After 6 h, the objects are placed contaminated side-down onto plates containing bacteria growth medium, then incubated at 37° C. for 12 h to allow any viable bacteria to grow. The presence or absence of bacterial growth indicates the efficacy of the composition on each surface.

### Example 12

### Efficacy of the Nanoscale Particle Antimicrobial Composition

The Nanoscale Particle Antimicrobial Compositions 1A, 2A, and 2C are tested against the avian influenza virus, Type A (H9N22), Turkey/Wis/66; SPAFAS through injection into embryonated chicken eggs. A virus suspension control is used for comparison purposes.

### Example 13

### Antimicrobial Treatment

Human Clinical Trial of the Safety and Efficacy of Nanoscale particle Antimicrobial Composition 1A, 2A or 2C to Prevent Recurrent Methicillin-Resistant Staphylococcus aureus (MRSA) Infection

Objective: The primary objective of this study is to evaluate the safety, local tolerability and efficacy of the nanoscale particle antimicrobial composition 1A, 2A or 2C. The composition is applied topically to subjects who are carriers of colonies of MRSA and MSSA. The extent of systemic absorption of the nanoscale particle antimicrobial composition is evaluated and the effect of the composition to clear colonies of MRSA.MSSA.

Study Design: This study is a randomized, double-blind, placebo-controlled, ascending dose Phase I/IIa study to evaluate the safety, tolerability and efficacy of topical nanoscale particle antimicrobial composition 1A, 2A or 2C in subjects colonized with methicillin-resistant/-sensitive Staphylococcus aureus (MRSA/MSSA). The first group of subjects receives the 1% nanoscale particle antimicrobial composition or placebo, the second group of subjects receives the 3% nanoscale particle antimicrobial composition or placebo, and the third group of subjects receives the 5% nanoscale particle antimicrobial composition or placebo. Dose escalation is performed after a brief safety evaluation of the tolerability after application of the nanoscale particle antimicrobial composition/placebo vehicle for three days. Pharmacokinetic samples are collected. Subjects are followed until 9 weeks after initiation of treatment.

Primary Outcome Measurements: explore safety and local tolerability and efficacy of nanoscale particle antimicrobial composition when applied topically to skin of subjects with colonized MRSA/MSSA; determine the extent of systemic absorption of nanoscale particle antimicrobial composition when applied to the skin of subjects. Secondary Outcome Measurements: to evaluate recurrence of MRSA/MSSA during the observation period (week 2 and week 9 after treatment).

### Example 14

### Antimicrobial Treatment

Human Clinical Trial of the Safety and Efficacy of Nanoscale particle Antimicrobial Composition 1A, 2A or 2C

Objective: The primary objective of this study is to measure the antimicrobial effectiveness of the nanoscale particle antimicrobial composition that live on the surface of the skin.

Study Design: This Phase III study is a non-randomized, single-center, open-label study. The study is comprised of 2 parts with approximately 20 subjects participating in each part. Subjects eligible for Part 1 have the nanoscale particle antimicrobial composition 1A, 2A or 2C applied to 6 sites across the chest and/or abdomen and chlorhexidine 2% solution (control, FDA approved medication) applied to 6 matching sites on the contralateral side. Swab cultures are obtained at specified time points over a period of 3 days. Subjects eligible for Part 2 each have nanoscale particle antimicrobial composition applied to 6 sites across the upper chest or abdomen. Swab cultures are obtained at specified time points over a period of 7 days. In addition, subjects in Part 2 have 2 peripheral catheters inserted, one in each arm. One catheter insertion site will be treated with nanoscale particle antimicrobial composition (following treatment with isopropyl alcohol) and the other site will be treated with chlorhexidine 2%/isopropyl alcohol. Swab cultures are obtained at specified time points over a period of 7 days.

Primary Outcome Measurements: change in mean number of skin bacterial counts from baseline to 73 hours; change in mean number of skin bacterial counts from baseline to 7 days, number of subjects with significantly colonized catheters, defined as greater than or equal to 15 colony forming units (CFUs) at 0 hours.

What is claimed is:

1. A formulation comprising: (a) one or more nanoscale particle having a particle size of about 5 nm to about 200 nm; and (b) a film-forming polymer.

2. The formulation of claim 1, wherein the film-forming polymer comprises polyolprepolymer-2 (PPG-12/SMDI), poly(styrene-co-maleic anhydride) copolymers (SMA), acrylate copolymers, cellulosic polymers, ethylene/acrylic acid copolymer, polyacrylic acid, C₁-C₅ alkyl galactomannan, isododecane/ethylene mixed copolymer, adipic acid/diethylene glycol/glycerin crosspolymer, trimethylpentanediol adipic acid copolymer, trimethylpentanediol/adipic acid/isononanoic acid, PVP/hexadecene copolymer, PVP/eicosene copolymer, alpha olefin/isopropyl maleate/MA polymer, cycloalkyl methacrylate copolymer/isododecane trimethyl polysiloxane, octadecene/MA copolymer, acrylates C₁₀-C₃₀ alkyl acrylate crosspolymer, cetyl hydroxyethylcellulose, dimethiconol, dimethicone, diglycol/cyclohexane-dimethanol/isophthalates/sulfoisophthalate copolymer, polyethylene, waxes, polyurethane, polyurethane resins, natural gums, or a combination thereof.

3. The formulation of claim 1, wherein the film-forming polymer is polyolprepolymer-2 (PPG-12/SMDI), poly(styrene-co-maleic anhydride) copolymers (SMA), polyurethane or a combination thereof.

4. The formulation of claim 1, wherein at least one nanoscale particle comprises silver, titanium, zinc, aluminum, iron, copper, platinum, zirconium, palladium, gold, salts thereof or a combination thereof.

5. The formulation of claim 4, wherein the silver nanoparticle comprises an ionic silver salt, elemental silver, or combinations thereof.

6. The formulation of claim 5, wherein the elemental silver is colloidal silver.

7. The formulation of claim 4, wherein the titanium is titanium dioxide.

8. The formulation of claim 1, further comprising a vehicle acceptable for topical administration.

9. The formulation of claim 1, wherein at least one nanoscale particle is present in a concentration of about 0.0000001% to about 5% by weight.

10. The formulation of claim 1, wherein the film-forming polymer is present in an amount of about 0.05 to about 5.0% by weight.

11. The formulation of claim 1, wherein the formulation provides immediate and sustained release of at least one nanoscale particle.

12. The formulation of claim 1, wherein the nanoscale particle has a particle size of about 100 to about 200 nm.

13. The formulation of claim 1, further comprising a coloring agent that adheres to the skin during use to indicate compliant application of product.

14. A method of reducing a population of pathogenic microorganisms on a surface comprising applying to the surface a formulation comprising: (a) one or more nanoscale particle having a particle size of about 5 nm to about 200 nm; and (b) a film-forming polymer.

15. The method of claim 14, wherein the nanoparticle comprises an ionic silver salt, elemental silver or combinations thereof.

16. The method of claim 14, wherein the film forming polymer is polyolprepolymer-2 (PPG-12/SMDI).

17. The method of claim 14, wherein the pathogenic microorganisms comprise bacteria, viruses, fungi, or combinations thereof.

18. The method of claim 14, wherein the pathogenic microorganisms comprise Aspergillus niger, Pseudomonas aeruginosa, Staphylococcus aureus (MRSA), Clostridium difficile, Vancomycin-resistant Enterococci, H1N1 influenza virus, or combinations thereof.

19. The method of claim 14, wherein the surface is non-porous or porous.

20. The method of claim 14, wherein the surface is a medical device.
